(19) United States
(12) Patent Application Publication
(10) Pub. No.: US 2024/0408117 A1
(43) Pub. Date: Dec. 12, 2024

(54) COMPOSITIONS AND METHODS FOR TREATING HEMIPLEGIC MIGRAINE

(71) Applicant: Schedule 1 Therapeutics, Inc., Chicago, IL (US)

(72) Inventor: George D. Pappas, Chicago, IL (US)

(73) Assignee: Schedule 1 Therapeutics, Inc., Chicago, IL (US)

(21) Appl. No.: 18/739,002

(22) Filed: Jun. 10, 2024

Related U.S. Application Data
(60) Provisional application No. 63/472,116, filed on Jun. 9, 2023.

(51) Int. Cl. A61K 31/00; A61K 45/06; A61P 25/06

(52) U.S. Cl. CPC A61K 31/658; A61K 45/06; A61P 25/06

(57) ABSTRACT

The present disclosure provides new pharmaceutical formulations comprising CBD and THC, their preparation, and their use for treating hemiplegic migraine.

### CROSS-REFERENCE TO RELATED APPLICATIONS

This application claims priority to U.S. Provisional Application No. 63/472,116, filed Jun. 9, 2023, the entire contents of which are incorporated by reference herein.

### FIELD OF THE DISCLOSURE

The present disclosure relates to new pharmaceutical formulations, their preparation, and their use for reducing acute migraine pain. In particular, the present invention relates to pharmaceutical formulations comprising CBD and THC.

### BACKGROUND OF THE INVENTION

Hemiplegic migraine (HM) is a very rare form of migraine characterized by unilateral weakness (paresis or paralysis of an arm or leg on both, on one side of the body). Other symptoms include headache, visual, sensory and/or speech/language dysfunction, repetitive daily blindness, permanent neurological features including recurrent coma, mental retardation, and other forms of cognitive impairment, epileptic attacks, and ataxia. HM shares characteristics with more common forms of migraine with aura (MA), although it distinct from MA in a number of ways. For instance, some of the attacks are characterized by unilateral weakness; paresis or paralysis of an arm or a leg or both one just one side of the body. This motor deficit may outlast the headache and could be accompanied by visual, sensory and/or speech/language dysfunction (Russell & Ducros, 2011). It is also distinguished by the severity of the condition. Repetitive daily blindness and permanent neurological features, including recurrent coma, mental retardation, and other forms of cognitive impairment, have been reported following HM attacks, along with epileptic attacks and ataxia. (Russell & Ducros, 2011) Furthermore, HM is distinguished from MA in pathophysiological characteristics such as responsiveness to known MA triggers. Both intravenous calcitonin gene-related peptide (CGRP) and nitric oxide trigger migraine-like attacks in patients with MA and migraine without aura (MO) but fail to trigger attacks in patients with FHM. (Hansen J. e., 2008) (Hansen J. M., 2011)

HM affects an estimated 0.005% of the US population (Thomsen L., 2002) and there are no FDA-approved therapeutics. Commonly prescribed acute care migraine medications, such as triptans (e.g., sumatriptan, naratriptan, zolmitriptan, etc.) are contraindicated. (Ong, 2018) The effects of recently approved acute care and preventive migraine treatments, such as gepants, lasmitidan, and monoclonal antibodies to either the CGRP ligand or its receptor, are unknown. (Russell & Ducros, 2011) However, intravenous CGRP, which triggers migraine-like attacks in patients with and without aura (MA/MO), fail to trigger attacks in patients with FHM, suggesting CGRP may not play an important role in HM. (Hansen J. e., 2008) (2010) (2011)

HM includes two subtypes: Familial Hemiplegic Migraine (FHM), in which the patient has at least a first- or second-degree relative that has migraine with aura and motor weakness, and Sporadic Hemiplegic Migraine (SHM) having similar clinical symptoms of migraine with aura and motor weakness with no first- or second-degree relatives with motor weakness. (International Classification of Headache Disorders, 3rd edition, 2018) At least 25% of the large families affected and most sporadic cases do not have a mutation in one of the three genes implicated, suggesting other, currently unknown, genes are involved. (Russell & Ducros, 2011)

The combination of disease severity and the risk of permanent neurological damage in the absence of known effective treatments targeting the underlying cause of HM underscore the significant unmet need for patients with this very rare disease.

### SUMMARY OF THE INVENTION

One embodiment of the present disclosure, described herein, is a pharmaceutical composition suitable for administration to a subject in need thereof comprising CBD and THC, wherein the weight ratio of CBD to THC is from about 10:1 to about 400:1.

In another embodiment of the present disclosure, described herein, is a pharmaceutical composition suitable for injection comprising CBD and THC, wherein the weight ratio of CBD to THC is from about 30:1 to about 300:1.

In another embodiment of the present disclosure described herein, is a pharmaceutical composition suitable for administration to a subject in need thereof comprising at least one minor cannabinoid, CBD, and THC.

In yet another embodiment of the present disclosure, described herein, is a pharmaceutical composition suitable for injection comprising at least one minor cannabinoid, CBD, and THC.

In still another embodiment of the present disclosure, described herein, is a method for treating acute migraine pain, the method comprising administering a pharmaceutical composition comprising CBD and THC to a subject in need thereof.

In still a further embodiment of the present disclosure, described herein, is a method for treating acute migraine pain, the method comprising administering a pharmaceutical composition comprising at least one minor cannabinoid, CBD, and THC to a subject in need thereof.

In any one of the proceeding embodiments of the present disclosure, the CBD and THC in the pharmaceutical composition are at least 90 wt. % pure.

In some aspects, the weight ratio of CBD to THC is from about 25:1 to about 400:1. In some aspects, the weight ratio of CBD to THC is from about 10:1 to about 300:1. In some aspects, the ratio of CBD to THC is from about 30:1 to about 100:1. In some additional aspects, In some additional aspects, the ratio of CBD to THC is from about 50:1 to about 150:1. In some additional aspects, the ratio of CBD to THC is from about 75:1 to about 125:1.

In some aspects, the ratio of CBD to THC is from 33.3:1. In some aspects, the ratio of CBD to THC is from 50:1. In some aspects, the ratio of CBD to THC is from 60:1. In some aspects, the ratio of CBD to THC is from 66.7:1. In some aspects, the ratio of CBD to THC is from 70:1. In some aspects, the ratio of CBD to THC is from 80:1. In some aspects, the ratio of CBD to THC is from 90:1. In some aspects, the ratio of CBD to THC is from 100:1. In some aspects, the ratio of CBD to THC is from 125:1. In some aspects, the ratio of CBD to THC is from 150:1. In some aspects, the ratio of CBD to THC is from 200:1.

In any one of the proceeding embodiments of the present disclosure, the pharmaceutical composition comprises 0.1% to 99% THC and 1% to 99.9% CBD.

In any one of the proceeding embodiments of the present disclosure, the pharmaceutical composition comprises a maximum dose of CBD of no more than 500 mg and comprises a maximum dose of THC of no more than 5 mg.

In any one of the proceeding embodiments of the present disclosure, the pharmaceutical composition further comprises a terpenoid. In some aspects, the terpenoid comprises myrcene, β-caryophyllene, limonene, α-terpineol, linalool, α-phellandrene, α-pinene, β-pinene, γ-terpinene, nerolidol, phytol, caryophyllene oxide, or α-humule.

In any one of the proceeding embodiments of the present disclosure, the pharmaceutical composition further comprises an analgesic agent. In some aspects, the analgesic agent comprises paracetamol, acetaminophen, a non-steroidal anti-inflammatory drug, a 5-HT (serotonin) receptor agonist, a calcitonin gene related peptide (CGRP) receptor antagonist, an N-methyl D-aspartate (NMDA) inhibitor, a glutamate receptor antagonist, a Substance P inhibitor, a GABA inhibitor, FAAH inhibitor, a Nerve Growth Factor (NGF) inhibitor, a Brain-Derived Neurotrophic Factor (BDNF) inhibitor, an extracellular signal-regulated kinase (ERK) antagonist, or a NO inhibitor.

In any one of the proceeding embodiments of the present disclosure, the pharmaceutical composition further comprises at least one minor cannabinoid. In some aspects, the at least one minor cannaboid is selected from a group consisting of CBG, CBGA, CBN, CBNA, THCV, THCA, CBC, CBCA, CBDV, and D8-THC. In some aspects, the at least one minor cannabinoid is at least 90 wt. % pure.

In any one of the proceeding embodiments of the present disclosure, the pharmaceutical composition further comprises a pharmaceutically acceptable excipient. In some aspects, the pharmaceutically acceptable excipient comprises a carrier, a diluent, a binder, a filler, a buffering agent, a pH modifying agent, a disintegrant, a dispersant, a preservative, a lubricant, a taste-masking agent, a flavoring agent, or a coloring agent.

In some aspects, the pharmaceutical composition is suitable for sublingual administration. In some aspects, the pharmaceutical composition is a liquid solution suitable for administration as a spray. In some aspects, the pharmaceutical composition is suitable for administration via injection. In some aspects, the pharmaceutical composition is suitable for administration by inhalation.

In some aspects, the pharmaceutical composition comprises an amount of CBD effective to reduce unwanted side effects of THC.

In yet a further embodiment of the present disclosure, described herein, is a method for treating acute migraine pain, the method comprising administering a pharmaceutical composition comprising at least one minor cannabinoid, CBD, and THC to a subject in need thereof.

In still another further embodiment of the present disclosure, described herein, is a method for treating acute migraine pain, the method comprising administering a pharmaceutical composition comprising at least one minor cannabinoid, CBD, and THC to a subject in need thereof.

In another further embodiment of the present disclosure, described herein, is a method for reducing acute migraine pain, the method comprising administering a pharmaceutical composition comprising at least one minor cannabinoid, CBD, and THC to a subject in need thereof.

In another further embodiment of the present disclosure, described herein, is a method for reducing spontaneous pain, the method comprising administering a pharmaceutical composition comprising at least one minor cannabinoid, CBD, and THC to a subject in need thereof.

In yet a further embodiment of the present disclosure, described herein, is a method for treating hemiplegic migraine comprising administering a pharmaceutical composition comprising at least one minor cannabinoid, CBD, and THC to a subject in need thereof.

In still a further embodiment of the present disclosure, described herein, is a method for treating familial hemiplegic migraine comprising administering a pharmaceutical composition comprising at least one minor cannabinoid, CBD, and THC to a subject in need thereof.

In yet a further embodiment of the present disclosure, described herein, is a method for treating sporadic hemiplegic migraine comprising administering a pharmaceutical composition comprising at least one minor cannabinoid, CBD, and THC to a subject in need thereof.

In some aspects, the pharmaceutical composition is administered upon first symptom onset of acute pain. In some aspects, the pharmaceutical composition is administered upon first symptom of spontaneous pain. In some aspects, the pharmaceutical composition is administered upon first symptom of hemiplegic migraine. In some aspects, the pharmaceutical composition is administered upon first symptom of familial hemiplegic migraine. In some aspects, the pharmaceutical composition is administered upon first symptom of sporadic hemiplegic migraine. In some aspects, a second dose of the pharmaceutical composition is administered 90 minutes or more after the first administration.

In some aspects, at least one biomarker of central and peripheral sensitization is modulated as a result of the administration. In some aspects, the at least one biomarker is selected from the group consisting of NMDA, glutamate, CGRP, FAAH, Substance P, 5-HT, NO, GABA, NGF, serotonin, dopamine, AEA, and 2-AG.

Other features and iterations of the invention are described in more detail below.

### BRIEF DESCRIPTION OF THE DRAWINGS

FIG. 1 shows the sagittal section of a Thy1-ChR2/YFP mouse, showing brain-wide YFP expression indicating global expression of ChR2 including cortical areas. Using blue light photo-stimulation, ChR2 channels are activated, resulting in neuronal depolarization, as a basis for optogenetic CSD induction used in Aims 1 and 2 described in Example 5.

FIG. 2 shows custom-made platinum-iridium electrodes for intracortical DC-EEG recordings used for Aim 1 CSD threshold measurements described in Example 5. Plastic 7-channel electrode socket contact shown on the left bottom.

FIG. 3 shows an example recording of DC-EEG (M1 on top, V1 below) and corresponding AC-EEG and (for V1) neuronal MUA, during CSD threshold induction by blue light photo-stimulation. The bottom trace shows blue LED pulses that were delivered at 4 mW intensity, using increasing pulse duration until a CSD occurred, as evident directly after the cessation of the last blue LED pulse. CSD is identified from i) large DC-shift (exceeding 5 mV), ii) spread from M1 to V1 (given the induction at M1 cortex), iii) suppression of AC-signal, and iv) suppression of MUA.

FIG. 4 shows placement of electrode (dots A-E; reference and ground on cerebellum) and optic fiber (dots X and Y) placement locations for Aim 1 experiments of Example 5. Note that optic fiber X served as backup in case no CSDs could be induced by photostimulation using optic fiber Y.

FIG. 5A shows CSD threshold measurements during daily or bi-daily baseline assessments performed in mice with a Diadent-based head-mount, that was coupled/uncoupled for each experimental day as detailed in Example 5. Already on the first day, 3 out of 6 mounts showed extremely high threshold values, requiring photo-stimulation power above 4 mW, or could not have a CSD induced at all. FIG. 5B shows the CSD threshold measurements using Sun Medical Cement, and keeping a mouse connected in the setup for the entire 3-4 week experimental series, which proved required for stable threshold measurements. Mice were only included for CBD:THC tests if baseline values tested over the course of 3 days proved stable.

FIGS. 6A-6C show example recordings of DC-EEG, AC-EEG, and neuronal multi-unit activity (MUA) during an optogenetic CSD induction paradigm as outlined in Example 5. The blue LED pulse on the bottom indicates the short photo-stimulation of the skull overlaying the primary motor cortex (M1) for optogenetic CSD induction. In this example, a CSD is induced as evident from the start of a large DC-deflection (exceeding 5 mV) in M1 cortex, spreading slowly, within tens of seconds up to a minute towards the V1 cortex. The CSD events are accompanied by a short peak followed by suppression of neuronal MUA, and suppression of the AC-EEG signal.

FIGS. 7A-7D shows the results of acute pretreatment of CSD with CBD and THC. Acute pretreatment with CBD:THC (administered 1 hour or 30 minutes prior to CSD threshold testing) did not affect opto-CSD threshold (A), propagation rate (B), half-width (C) or amplitude (D) for either 100:1 or 30:1 dosages in WT or FHM1 mice. Half-width and amplitude were assessed separately for CSD events in the primary motor cortex (M1) and visual cortex (V1). All p-values>0.05 for the comparison to vehicle treatment.

FIG. 8 shows graphs measuring mouse grimace scale (MGS) in wild type and FHM1 mice. Optogenetically induced CSD caused an increase in MGS in the 30-40 minutes window after the first CSD in freely behaving WT and FHM1 mice (indicated as ‘30 min’ post-CSD). This short-term opto-CSD-induced rise in MGS is evident at 30 minutes had recovered to baseline levels at 24 hours after CSD, both in WT and FHM1 mice. Data shown are combined data from untreated and vehicle experiments from N=6 WT and N=7 FHM1 mice.

FIG. 9 shows graphs measuring mouse grimace scale (MGS) in wild type and FHM1 mice after acute pretreatment with CBD and THC. Acute pretreatment by CBD:THC (administered 30 minutes prior to the first of 3 optogenetically induced CSD events) prevents the CSD-induced increase in MGS score, as assessed in the 30-40 minute window following the first CSD, both for 100:1 and 30:1 dosages (p-values in the plot are for combined WT and FHM1 data).

### DETAILED DESCRIPTION OF THE INVENTION

Before the present invention is disclosed and described, it is to be understood that this invention is not limited to the particular methods, compositions, or materials disclosed herein, but is extended to equivalents thereof as would be recognized by those ordinarily skilled in the relevant arts. It should also be understood that terminology employed herein is used for the purpose of describing particular embodiments only and is not intended to be limiting.

Described herein are compositions suitable for administration to a subject in need thereof comprising CBD and THC, wherein the weight ratio of CBD to THC is from about 10:1 to about 400:1. The pharmaceutical composition may further comprise a terpenoid, an analgesic agent, at least one minor cannabinoid, or a pharmaceutically acceptable excipient comprising a carrier, a diluent, a binder, a filler, a buffering agent, a pH modifying agent, a disintegrant, a dispersant, a preservative, a lubricant, a taste-masking agent, a flavoring agent, a coloring agent, or any combination thereof.

### I. Pharmaceutical Compositions Comprising CBD and THC

In one aspect, the present disclosure encompasses a pharmaceutical composition comprising CBD (cannabidiol) and THC (D9-THC or dronabinol), wherein the weight ratio of CBD to THC is from about 10:1 to about 400:1.

The pharmaceutical composition comprising CBD (cannabidiol) and THC (D9-THC or dronabinol) comprises a liquid solution. The pharmaceutical compositions, described herein, may be suitable for administration to a subject in need thereof. Suitable methods of administration may be sublingual administration as a liquid solution which may be sprayed into the oral cavity of the subject in need thereof, inhalation administration as a spray, or injection of the liquid solution.

In one embodiment, the pharmaceutical composition comprises CBD and THC. In another embodiment, the pharmaceutical composition comprises CBD alone. In still another embodiment, the pharmaceutical composition comprises THC alone.

In general, the CBD and THC each are at least 90 wt. % pure. In various embodiments, the at least one minor cannabinoid is at least 90 wt. % pure, at least 91 wt. % pure, at least 92 wt. % pure, at least 93 wt. % pure, at least 94 wt. % pure, at least 95 wt. % pure, at least 96 wt. % pure, at least 97 wt. % pure, at least 98 wt. % pure, at least 99 wt. % pure, or at least 99.9 wt. % pure. The CBD and the THC may each be derived from plants or may be synthetically produced.

The pharmaceutical composition comprises a weight ratio of CBD and THC. Generally, the weight ratio of CBD and THC may range from about 10:1 to about 400:1. In various embodiments, the weight ratio of CBD to THC may be about 10:1, about 15:1, about 20:1, about 25:1, about 30:1, about 35:1, about 40:1, about 45:1, about 50:1, about 55:1, about 60:1, about 70:1, about 80:1, about 85:1, about 90:1, about 95:1, about 100:1, about 105:1, about 110:1, about 115:1, about 120:1, about 125:1, about 130:1, about 135:1, about 140:1, about 145:1, about 150:1, about 155:1, about 160:1, about 165:1, about 170:1, about 175:1, about 180:1, about 185:1, about 190:1, about 195:1, about 200:1, about 205:1, about 210:1, about 215:1, about 220:1, about 225:1, about 230:1, about 235:1, about 240:1, about 245:1, about 250:1, about 255:1, about 260:1, about 265:1, about 270:1, about 275:1, about 280:1, about 285:1, about 290:1, about 295:1, about 300:1, about 305:1, about 310:1, about 320:1, about 325:1, about 330:1, about 335:1, about 340:1, about 345:1, about 350:1, about 355:1, about 360:1, about 365:1, about 370:1, about 375:1, about 380:1, about 385:1, about 390:1, about 395:1, or about 400:1. In various embodiments, the weight ratio of CBD and THC may range from about 10:1 to about 400:1, from about 10:1 to about 25:1, from about 25:1 to about 50:1, from about 50:1 to about 75:1, from about 75:1 to about 100:1, from about 75:1 to about 100:1, from about 100:1 to about 125:1, from about 125:1 to about 150:1, from about 150:1 to about 175:1, from about 175:1 to about 200:1, from about 200:1 to about 225:1, from about 225:1 to about 250:1, from about 250:1 to about 275:1, from about 275:1 to about 300:1, from about 300:1 to about 325:1, from about 325:1 to about 350:1, from about 350:1 to about 375:1, from about 375:1 to about 400:1, from about 25:1 to about 400:1, from about 10:1 to about 300:1, from about 30:1 to about 300:1, from about 33.3:1 to about 200:1, from about 50:1 to about 150:1, from about 60:1 to about 125:1, from about 75:1 to about 125:1, from about 66.7:1 to about 100:1, from about 70:1 to about 90:1, or from about 75:1 to about 85:1.

In general, the pharmaceutical composition may comprise from about 0.1% to about 99% THC by weight. In various embodiments, pharmaceutical composition may comprise about 0.1% to about 99% of THC, such as about 0.1%, about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% THC by weight of the pharmaceutical composition. In various embodiments, the pharmaceutical composition may comprise from about 0.1% to about 10%, from about 10% to about 20%, from about 20% to about 30%, from about 30% to about 40%, from about 40% to about 50%, from about 50% to about 60%, from about 60% to about 70%, from about 70% to about 80%, from about 80% to about 90%, from about 90% to about 99%, from about 0.1% to about 20%, from about 20% to about 40%, from about 40% to about 60%, from about 60% to about 80%, from about 80% to about 99%, from about 0.1% to about 50%, or from about 50% to about 99% THC by weight of the pharmaceutical composition.

Generally, the pharmaceutical composition may comprise from about 1% to about 99.9% CBD by weight of the pharmaceutical composition. In various embodiments, pharmaceutical composition may comprise about 1% to about 99.9% of CBD by weight of the pharmaceutical composition, such as about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 99.9% CBD by weight of the pharmaceutical composition. In various embodiments, the pharmaceutical composition may comprise from about 1% to about 10%, from about 10% to about 20%, from about 20% to about 30%, from about 30% to about 40%, from about 40% to about 50%, from about 50% to about 60%, from about 60% to about 70%, from about 70% to about 80%, from about 80% to about 90%, from about 90% to about 99.9%, from about 1% to about 20%, from about 20% to about 40%, from about 40% to about 60%, from about 60% to about 80%, from about 80% to about 99.9%, from about 1% to about 50%, or from about 50% to about 99.9% CBD by weight of the pharmaceutical composition.

The pharmaceutical composition may comprise a maximum dose of CBD of no more than about 500 mg. In various embodiments, the pharmaceutical composition may comprise a maximum dose of CBD of about 250 mg, about 300 mg, about 350 mg, about 400 mg, about 450 mg, or about 500 mg but no more than about 500 mg.

The pharmaceutical composition may comprise a maximum dose of THC of no more than about 5 mg. In various embodiments, the pharmaceutical composition may comprise a maximum dose of THC of about 1 mg, about 2 mg, about 3 mg, about 4 mg, or about 5 mg but no more than about 5 mg.

### Minor Cannabinoid

The pharmaceutical composition may further comprise at least one minor cannabinoid. The at least one minor cannabinoid may be selected from the group consisting of CBG (cannabigerol), CBGA (cannabigerolic Acid), CBN (cannabinol), CBNA (cannabinolic acid), THCV (tetrahydrocannabivarin), THCA (tetrahydrocannabinolic acid), CBC (cannabichromene), CBCA (cannabichromene acid), CBDV (cannabidivarin), D8-THC (D8-tetrahydrocannabinol), and any combination thereof.

In general, the at least one minor cannabinoid may be at least 90 wt. % pure. In various embodiments, the at least one minor cannabinoid may be at least 90 wt. % pure, at least 91 wt. % pure, at least 92 wt. % pure, at least 93 wt. % pure, at least 94 wt. % pure, at least 95 wt. % pure, at least 96 wt. % pure, at least 97 wt. % pure, at least 98 wt. % pure, at least 99 wt. % pure, or at least 99.9 wt. % pure. The at least one minor cannabinoid may be plant-derived or may be synthetically produced.

Generally, the pharmaceutical composition may comprise the at least one minor cannabinoid in an amount from about 1% to about 99% by weight, such as about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% % by weight of the pharmaceutical composition. In various embodiments, the pharmaceutical composition may comprise the at least one minor cannabinoid in an amount from about 1% to about 10%, from about 10% to about 20%, from about 20% to about 30%, from about 30% to about 40%, from about 40% to about 50%, from about 50% to about 60%, from about 60% to about 70%, from about 70% to about 80%, from about 80% to about 90%, from about 90% to about 99.9%, from about 1% to about 20%, from about 20% to about 40%, from about 40% to about 60%, from about 60% to about 80%, from about 80% to about 99%, from about 1% to about 50%, or from about 50% to about 99.9% by weight of the pharmaceutical composition.

### Terpenoid

The pharmaceutical composition may further comprise a terpenoid. The terpenoid may comprise myrcene, β-caryophyllene, limonene, α-terpineol, linalool, α-phellandrene, α-pinene, β-pinene, γ-terpinene, nerolidol, phytol, caryophyllene oxide, or α-humule, other terpenoids known in the art, or a combination thereof.

In general, the pharmaceutical composition may comprise a terpenoid in an amount from about 1% to about 99% by weight, such as about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% % by weight of the pharmaceutical composition. In various embodiments, the pharmaceutical composition may comprise the terpenoid in an amount from about 1% to about 10%, from about 10% to about 20%, from about 20% to about 30%, from about 30% to about 40%, from about 40% to about 50%, from about 50% to about 60%, from about 60% to about 70%, from about 70% to about 80%, from about 80% to about 90%, from about 90% to about 99.9%, from about 1% to about 20%, from about 20% to about 40%, from about 40% to about 60%, from about 60% to about 80%, from about 80% to about 99%, from about 1% to about 50%, or from about 50% to about 99.9% by weight of the pharmaceutical composition.

### Analgesic Agent

The pharmaceutical composition may further include an analgesic agent. The analgesic agent may comprise paracetamol, acetaminophen, a non-steroidal anti-inflammatory (NSAID) drug, a 5-HT (serotonin) receptor agonist, a calcitonin gene related peptide (CGRP) receptor antagonist, an N-methyl D-aspartate (NMDA) inhibitor, a glutamate receptor antagonist, a Substance P inhibitor, a GABA inhibitor, FAAH inhibitor, a Nerve Growth Factor (NGF) inhibitor, a Brain-Derived Neurotrophic Factor (BDNF) inhibitor, an extracellular signal-regulated kinase (ERK) antagonist, a NO inhibitor, or a combination thereof.

In general, the pharmaceutical composition may comprise the analgesic agent in an amount from about 1% to about 99% by weight of the pharmaceutical composition, such as about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% % by weight of the pharmaceutical composition. In various embodiments, the pharmaceutical composition may comprise an analgesic agent in an amount from about 1% to about 10%, from about 10% to about 20%, from about 20% to about 30%, from about 30% to about 40%, from about 40% to about 50%, from about 50% to about 60%, from about 60% to about 70%, from about 70% to about 80%, from about 80% to about 90%, from about 90% to about 99.9%, from about 1% to about 20%, from about 20% to about 40%, from about 40% to about 60%, from about 60% to about 80%, from about 80% to about 99%, from about 1% to about 50%, or from about 50% to about 99.9% by weight of the pharmaceutical composition.

### Pharmaceutically Acceptable Excipient

The pharmaceutical composition may further include a pharmaceutical acceptable excipient. Non-limiting examples of pharmaceutically acceptable excipients include carriers, diluents, binders, fillers, buffering agents, pH modifiers, disintegrants, dispersants, preservatives, lubricants, sweetening agents, taste masking agents, flavoring agents, thickening agents, and combinations thereof. These excipients do not deleteriously react with the composition.

In one aspect, the pharmaceutically acceptable excipient may include a carrier. A carrier may be aqueous, organic, inorganic, or any combination thereof. Non-limiting examples of a carrier include water, polyethylene glycol, propylene glycol, ethanol, corn oil, cottonseed oil, peanut oil, sesame oil, benzyl alcohol, protein carriers, lipids, aqueous sodium chloride, agar, agaropectin, xanthan gum, guar gum, liposomes, niosomes, transferosomes, glycerin, and various buffers.

In one aspect, the pharmaceutically acceptable excipient may include a diluent. A diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated (phosphorylated) corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

In another aspect, the pharmaceutically acceptable excipient may include a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, polypeptides, oligopeptides, and combinations thereof.

In another aspect, the pharmaceutically acceptable excipient may include a filler. Suitable fillers include, but are not limited to carbohydrates, inorganic compounds, and polyvinylpyrrolidone. By way of non-limiting example, the filler may include calcium sulfate (both di- and tri-basic), starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

In still another aspect, the pharmaceutically acceptable excipient may include a buffering agent. Representative examples of suitable buffering agents include, but are not limited to, phosphates, carbonates, citrates, tris buffers, and buffered saline salts (e.g., Tris buffered saline or phosphate buffered saline).

In various aspects, the pharmaceutically acceptable excipient may include a pH modifier to adjust the pH of the formulation to a desired level. By way of non-limiting example, the pH modifier may include sodium carbonate, sodium bicarbonate, sodium citrate, citric acid, trisodium citrate, or phosphoric acid. In general, the pH of the composition may be either basic or acidic.

In a further aspect, the pharmaceutically acceptable excipient may include a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth. Non-limiting examples of suitable effervescent disintegrants include sodium bicarbonate in combination with citric acid and sodium bicarbonate in combination with tartaric acid.

In yet another aspect, the pharmaceutically acceptable excipient may include a dispersant or dispersing enhancing agent. Suitable dispersants include, but are not limited to starch, alginic acid, polyvinylpyrrolidones, guar gum, kaolin, bentonite, purified wood cellulose, sodium starch glycolate, isoamorphous silicate, and microcrystalline cellulose.

In still another aspect, the pharmaceutically acceptable excipient may include a preservative. Non-limiting examples of suitable preservatives include antioxidants, such as BHA, BHT, vitamin A, vitamin C, vitamin E, or retinyl palmitate, sodium benzoate, trisodium citrate, citric acid, sodium citrate; chelators such as EDTA or EGTA; and antimicrobials, such as parabens, chlorobutanol, or phenol.

In a further aspect, the pharmaceutically acceptable excipient may include a lubricant. Non-limiting examples of suitable lubricants include minerals such as talc or silica; and fats such as vegetable stearin, magnesium stearate or stearic acid.

In another aspect, the pharmaceutically acceptable excipient may include a sweetening agent. Non-limiting examples of sweetening agents include sucralose, saccharin, aspartame, mannitol, sorbitol, sucrose, maltose, fructose, lactose, xylitol, or combinations thereof. In one aspect, the sweetening agent is sucralose.

In yet another aspect, the pharmaceutically acceptable excipient may include a taste-masking agent. Taste-masking materials include cellulose ethers; polyethylene glycols; polyvinyl alcohol; polyvinyl alcohol and polyethylene glycol copolymers; monoglycerides or triglycerides; acrylic polymers; mixtures of acrylic polymers with cellulose ethers; cellulose acetate phthalate; citric acid; and combinations thereof.

In an alternate aspect, the pharmaceutically acceptable excipient may include a flavoring agent or bitterness masking agent. A flavoring agent may be chosen from synthetic flavor oils and flavoring aromatics and/or natural oils, extracts from plants, leaves, flowers, fruits, maltodextrin, hydroxypropyl, and combinations thereof. Non-limiting exemplary flavors are orange, lemon, lime, lemon-lime, lemonade, cherry (including sour cherry and black cherry), passion fruit, strawberry, blueberry, raspberry, mixed berry, and grape.

In another aspect, the pharmaceutically acceptable excipient may include a thickening agent. Non-limiting examples of these components are xanthan gum, guar gum, poloxamer, pectin, agar, gelatin, salts of alginic acid, carrageenan locust bean gum, and any combination thereof. By way of non-limiting example, a thickening agent is xanthan gum.

In still a further aspect, the pharmaceutically acceptable excipient may include a coloring agent. Suitable color additives include, but are not limited to, food, drug and cosmetic colors (FD&C), drug and cosmetic colors (D&C), or external drug and cosmetic colors (Ext. D&C).

### II. Methods for Preparing a Pharmaceutical Composition Comprising CBD and THC

Another aspect of the present disclosure encompasses a method for preparing a pharmaceutical composition comprising CBD and THC. The methods generally comprise combining CBD and THC forming a mixture; optionally adding a pharmaceutically acceptable excipient, a terpenoid, or an analgesic agent to the mixture. The methods are operable to produce any pharmaceutical composition described in Section (I).

### Combining CBD and THC

The method initiates be combining CBD and THC. CBD and THC are described in more detail in above in Section (I). CBD and THC may be added in any sequential order, in portions, or all at once.

CBD and THC may be mixed using various mean known in the art such as magnetic stirring or mechanical stirring.

This step in the method may be conducted at a temperature that ranges from about 0° C. to about 50° C. In various embodiments, the temperature may range from about 0° C. to about 50° C., from about 10° C. to about 40° C., or from about 20° C. to about 30° C. In one embodiment, the combining may be conducted at temperature of about 23° C. (i.e., room temperature). The combining typically is performed under ambient pressure. The combining may also be conducted under an inert atmosphere, for example under nitrogen, argon, helium, or a combination thereof.

Generally, this combining step is allowed to proceed for a sufficient period of time until the mixture is well-mixed. Whether the CBD and THC are well dissolved may be determined visually. Those having ordinary skill in the art are capable of visually determining when the CBD and THC are well dissolved. The duration of the combining step may range from about 5 minutes to about 2 hours. In some embodiments, the duration of the combining may range from about 5 minutes to about 30 minutes, from about 30 minutes to about 1 hours, or from about 1 hour to about 2 hours.

### Optionally Adding a Pharmaceutically Acceptable Excipient

The next step in the method is an optional step. This step comprises adding a pharmaceutically acceptable excipient to the mixture of the CBD and THC formed in the previous step. The pharmaceutically acceptable excipient may be added in portions or all at once.

Pharmaceutically Acceptable Excipients suitable for use in the present method are described in more detail above in Section (I).

This optional step in the method may be conducted at a temperature that ranges from about 0° C. to about 50° C. In various embodiments, the temperature may range from about 0° C. to about 50° C., from about 10° C. to about 40° C., or from about 20° C. to about 30° C. In one embodiment, the combining may be conducted at temperature of about 23° C. (i.e., room temperature). The optional step typically is performed under ambient pressure. The optional step may also be conducted under an inert atmosphere, for example under nitrogen, argon, helium, or a combination thereof.

Generally, this optional step is allowed to proceed for a sufficient period of time until the mixture is well-mixed. Whether the pharmaceutically acceptable excipient is well dissolved in the mixture may be determined visually. Those having ordinary skill in the art are capable of visually determining when the pharmaceutically acceptable excipient is well dissolved. The duration of the optional step may range from about 5 minutes to about 2 hours. In some embodiments, the duration of the optional step may range from about 5 minutes to about 30 minutes, from about 30 minutes to about 1 hours, or from about 1 hour to about 2 hours.

### Optionally Adding an Analgesic Agent

The next step in the method is an optional step. This step comprises adding an analgesic agent to the mixture of the CBD and THC formed in the previous step. The analgesic agent may be added in portions or all at once.

Analgesic agents suitable for use in the present method are described in more detail above in Section (I).

This optional step in the method may be conducted at a temperature that ranges from about 0° C. to about 50° C. In various embodiments, the temperature may range from about 0° C. to about 50° C., from about 10° C. to about 40° C., or from about 20° C. to about 30° C. In one embodiment, the combining may be conducted at temperature of about 23° C. (i.e., room temperature). The optional step typically is performed under ambient pressure. The optional step may also be conducted under an inert atmosphere, for example under nitrogen, argon, helium, or a combination thereof.

Generally, this optional step is allowed to proceed for a sufficient period of time until the mixture is well-mixed. Whether the analgesic agent is well dissolved in the mixture may be determined visually. Those having ordinary skill in the art are capable of visually determining when the analgesic agent is well dissolved. The duration of the optional step may range from about 5 minutes to about 2 hours. In some embodiments, the duration of the optional step may range from about 5 minutes to about 30 minutes, from about 30 minutes to about 1 hours, or from about 1 hour to about 2 hours.

### Optionally Adding a Terpenoid

The next step in the method is an optional step. This step comprises adding a terpenoid to the mixture of the CBD and THC formed in the previous step. The terpenoid may be added in portions or all at once.

Terpenoids for use in the present method are described in more detail above in Section (I).

This optional step in the method may be conducted at a temperature that ranges from about 0° C. to about 50° C. In various embodiments, the temperature may range from about 0° C. to about 50° C., from about 10° C. to about 40° C., or from about 20° C. to about 30° C. In one embodiment, the combining may be conducted at temperature of about 23° C. (i.e., room temperature). The optional step typically is performed under ambient pressure. The optional step may also be conducted under an inert atmosphere, for example under nitrogen, argon, helium, or a combination thereof.

Generally, this optional step is allowed to proceed for a sufficient period of time until the mixture is well-mixed. Whether the terpenoid is well dissolved in the mixture may be determined visually. Those having ordinary skill in the art are capable of visually determining when the terpenoid is well dissolved. The duration of the optional step may range from about 5 minutes to about 2 hours. In some embodiments, the duration of the optional step may range from about 5 minutes to about 30 minutes, from about 30 minutes to about 1 hours, or from about 1 hour to about 2 hours.

### III. Methods of Treatment

Further provided herein are methods for treating hemiplegic migraine by administering a pharmaceutical composition of the present disclosure to a subject in need thereof. In an aspect, the present disclosure encompasses a method for reducing or treating acute pain of migraine attacks as compared to a placebo. The method comprises administering a pharmaceutical composition comprising CBD and THC as a liquid to a subject in need thereof. In general, the pharmaceutical composition may be administered sublingually, intranasally, intradermally, subcutaneously, or intramuscularly. In one embodiment, the pharmaceutical composition may be administered sublingually. In another embodiment, the pharmaceutical composition may be administered intranasally. In yet another embodiment, the pharmaceutical composition may be administered intradermally. In still another embodiment, the pharmaceutical composition may be administered subcutaneously. In another embodiment, the pharmaceutical composition may be administered intramuscularly.

The reduction or treatment of acute pain of migraine attacks may be measured in a four-point Likert scale. The Likert scale may be used for headache pain (i.e., 0=none, 1=mild, 2=moderate, 3-severe), and a binary scale (present or absent). The Likert scale may be also used for associated migraine symptoms. In an endpoint, the reduction or treatment of hemiplegic may be the freedom of hemiplegia, the freedom of arm hemiplegia severity, the freedom from leg hemiplegia severity, and the freedom from any hemiplegic severity. The freedom from hemiplegia may be at 15 minutes, 30 minutes, 45 minutes, 1 hour, 2 hours, 4 hours, 24 hours, and 48 hours. For arm hemiplegia, using the Likert scale, on a 0 to 3 scale, with 3 indicating not able to move arm, 2 indicating able to move arm but not hold item, 1 indicating able to hold item but not lift arm overhead, and 0 indicating able to hold item and lift arm overhead, the improvement may be determined. For leg hemiplegia, on a scale 0 to 3, with 0 indicating not able to move leg, 1 indicating able to move leg but not stand, 2 indicating able to stand but not able to walk, and 3 indicating able to walk, the improvement may be determined.

The reduction or treatment of acute pain of migraine attacks may also provide other endpoints based on prior randomized controlled trial (RCTs) of cannabis for the treatment of pain. A subject may be asked to report euphoria, sleepiness, impairment, and highness. Euphoria, as defined herein, may be a pleasant subjective feeling associated with drug liking. The term highness, as defined herein, can be used to describe subjective impairment that may or may not be pleasant and may not be associated with drug liking.

Generally, the acute pain may be caused by familial hemiplegic migraine, acute hemiplegic migraine, sporadic hemiplegic migraine, or a combination thereof. In various embodiments, the familial hemiplegic migraine may be familial hemiplegic migraine type 1, familial hemiplegic migraine type 2, or familial hemiplegic migraine type 3. The methods may include first confirming the hemiplegic migraine genotype of the subject. Confirming the hemiplegic migraine genotype of the subject may comprise determining the presence or level of one or more biomarkers associated with hemiplegic migraine in a sample of a bodily fluid collected from the subject.

The pharmaceutical compositions comprising CBD and THC may be any of the compositions described in Section (I).

The subject may be a human. The human may be a male or a female from 18-75 years of age. The human may be subject to chronic migraines, may have a high frequency of migraines, or may demonstrate a facial grimace as a result of migraine pain. The human may have at least 4 migraines per month, able to provide informed consent, agree not to use cannabis outside of the study during the participation of the study, and agree not to drive a motor vehicle or operate heavy machinery within 4 hours following last use of cannabis during participation in the study.

### Administration

In general, the pharmaceutical composition is administered within 5 minutes of the first symptom onset of acute pain caused by hemiplegic migraine. In various embodiments, the pharmaceutical composition is administered within 5 minutes, within 10 minutes, within 30 minutes, within 1 hour, within 2 hours, or within 4 hours of the first symptom onset of acute pain caused by hemiplegic migraine.

The pharmaceutical composition comprising CBD and THC may be administered topically, parentally, sublingually, intranasally, or intradermally. Sublingual or intranasal administration may be accomplished by using a spray container that emits a spray of the pharmaceutical composition as a mist. Such spray containers and methods of making, procuring, and using the same are generally known in the pharmaceutical arts. Sublingual administration may be also placing the liquid pharmaceutical composition under the tongue without chewing or swallowing. Topical administration may also involve the use of transdermal administration such as transdermal patches or iontophoresis devices. The term “parenteral” as used herein includes subcutaneous, intravenous, intramuscular, or intrasternal injection, or infusion techniques.

Generally, if the acute pain caused by the hemiplegic migraine persists, a second dose of the pharmaceutical composition comprising CBD and THC may be administered 90 minutes or more after the first administration. In various embodiments, if the acute pain caused by the hemiplegic migraine persists, a second dose of the pharmaceutical composition comprising CBD and THC may be administered 90 minutes or more, 120 minutes or more, 150 minutes or more, 180 minutes or more, 210 minutes or more, or 240 minutes or more after the first administration. However, in some aspects, no more than 2 administrations are performed within a 24-hour period.

In general, the treatment for acute migraine pain may be effective within at least two hours after administration. In various embodiments, treatment for acute migraine pain may be effective within at least two hours, within at least three hours, within at least four hours, within at least twelve hours, within at least twenty-four hours, within at least thirty-six hours, or within at least forty hours after administration.

Effects of the Pharmaceutical Composition after Administration

After administration, at least one biomarker of central and peripheral sensitization may be modulated (i.e., increased or decreased). The at least one biomarker may be selected from the group consisting of N-methyl-D-aspartate (NMDA), glutamate, calcitonin gene-related peptide (CGRP), fatty acid amide hydrolase (FAAH), modulator of pain perception (Substance P), 5-hydroxytryptamine (5-HT), nitric oxide (NO), γ-aminobutyric acid (GABA), nerve growth factor (NGF), serotonin, dopamine, anandamide (AEA), and 2-arachidonoylglycerol (2-AG). Generally, the at least one biomarker of central and peripheral sensitization may be measured by a serum concentration of a blood sample.

The pharmaceutical composition comprises an amount of CBD effective to reduce unwanted side effects of THC. The unwanted side effects of THC may include psychoactivity, psychosis, anxiety, paranoia, dizziness, confusion, cardiovascular risk, memory impairment, or combinations thereof.

Administering the pharmaceutical composition may reduce the intensity, duration, or frequency of one or more symptoms of hemiplegic migraine. Symptoms of hemiplegic migraine include hemiplegia (including leg hemiplegia and arm hemiplegia), hemiparesis, headache pain, photophobia, nausea, and other symptoms of hemiplegic migraine known in the art.

Generally, the subject may experience reduced headache pain within 15 minutes of the administration. In various embodiments, the subject may experience reduced headache pain within 15 minutes, within 30 minutes, within 1 hour, within 4 hours, within 24 hours, or within 48 hours of the administration.

In general, the subject may be free of headache pain within 15 minutes of the administration. In various embodiments, the subject may be free of headache pain within 15 minutes, within 30 minutes, within 1 hour, within 4 hours, within 24 hours, or within 48 hours of the administration.

In general, the subject may experience reduced photophobia within 15 minutes of the administration. In various embodiments, the subject may experience reduced photophobia within 15 minutes, within 30 minutes, within 1 hour, within 4 hours, within 24 hours, or within 48 hours of the administration.

In general, the subject may be free of photophobia within 15 minutes of the administration. In various embodiments, the subject may be free of photophobia within 15 minutes, within 30 minutes, within 1 hour, within 4 hours, within 24 hours, or within 48 hours of the administration.

Generally, the subject may experience reduced nausea within 15 minutes of the administration. In various embodiments, the subject may experience reduced nausea within 15 minutes, within 30 minutes, within 1 hour, within 4 hours, within 24 hours, or within 48 hours of the administration.

In general, the subject may be free of nausea within 15 minutes of the administration. In various embodiments, the subject may be free of nausea within 15 minutes, within 30 minutes, within 1 hour, within 4 hours, within 24 hours, or within 48 hours of the administration.

In general, the subject may be free of hemiplegia within 15 minutes of the administration. In various embodiments, the subject may be free of hemiplegia within 15 minutes, within 30 minutes, within 1 hour, within 4 hours, within 24 hours, or within 48 hours of the administration.

Generally, the subject may experience reduced arm hemiplegia within 15 minutes of the administration. In various embodiments, the subject may experience reduced arm hemiplegia within 15 minutes, within 30 minutes, within 1 hour, within 4 hours, within 24 hours, or within 48 hours of the administration.

In general, the subject may experience reduced leg hemiplegia within 15 minutes of the administration. In various embodiments, the subject may experience reduced leg hemiplegia within 15 minutes, within 30 minutes, within 1 hour, within 4 hours, within 24 hours, or within 48 hours of the administration.

Generally, the subject may experience reduced hemiparesis within 15 minutes of the administration. In various embodiments, the subject may experience reduced hemiparesis within 15 minutes, within 30 minutes, within 1 hour, within 4 hours, within 24 hours, or within 48 hours of the administration.

The pharmaceutical composition may be administered prior to the onset of hemiplegic migraine symptoms. In such embodiments, the pharmaceutical composition may be administered up to one hour prior to the onset of hemiplegic migraine symptoms, up to 30 minutes prior to the onset of hemiplegic migraine symptoms, up to 15 minutes prior to the onset of hemiplegic migraine symptoms, up to 10 minutes prior to the onset of hemiplegic migraine symptoms, or up to 5 minutes prior to the onset of hemiplegic migraine symptoms.

When administered prior to the onset of hemiplegic migraine symptoms, the administration may result in reduced intensity of the hemiplegic migraine symptoms, reduced duration of hemiplegic migraine symptoms, reduced frequency of hemiplegic migraine symptoms, reduced frequency of hemiplegia attacks, reduced intensity of hemiplegia attacks, reduced duration of hemiplegia attacks, etc.

In some embodiments, the pharmaceutical composition may be administered to treat or prevent epilepsy in the subject. Epilepsy and hemiplegic migraine have similar genetic relationships. Accordingly, administering a pharmaceutical composition of the present disclosure to a subject in need thereof may reduce the severity, frequency, or duration of symptoms of epilepsy (e.g., epileptic seizure) or may prevent the onset of symptoms of epilepsy when administered prior to the onset of epileptic symptoms.

### ENUMERATED EMBODIMENTS

Embodiment 1: A pharmaceutical composition suitable for administration to a subject in need thereof comprising CBD and THC, wherein a weight ratio of CBD to THC is from about 10:1 to about 400:1.

Embodiment 2: The pharmaceutical composition of embodiment 1, wherein the THC and CBD are at least 90 wt. % pure.

Embodiment 3: The pharmaceutical composition of embodiment 1 or 2, wherein the weight ratio of CBD to THC is from about 30:1 to about 300:1.

Embodiment 4: The pharmaceutical composition of any one of embodiments 1-3, wherein the ratio of CBD to THC is from about 25:1 to about 400:1.

Embodiment 5: The pharmaceutical composition of any one of embodiments 1-4, wherein the ratio of CBD to THC is from about 30:1 to about 100:1.

Embodiment 6: The pharmaceutical composition of any one of embodiments 1-5, wherein the ratio of CBD to THC is about 33.3:1.

Embodiment 7: The pharmaceutical composition of any one of embodiments 1-5, wherein the ratio of CBD to THC is about 50:1.

Embodiment 8: The pharmaceutical composition of any one of embodiments 1-5, wherein the ratio of CBD to THC is about 60:1.

Embodiment 9: The pharmaceutical composition of any one of embodiments 1-5, wherein the ratio of CBD to THC is about 66.7:1.

Embodiment 10: The pharmaceutical composition of any one of embodiments 1-5, wherein the ratio of CBD to THC is about 70:1.

Embodiment 11: The pharmaceutical composition of any one of embodiments 1-5, wherein the ratio of CBD to THC is about 80:1.

Embodiment 12: The pharmaceutical composition of any one of embodiments 1-5, wherein the ratio of CBD to THC is about 90:1.

Embodiment 13: The pharmaceutical composition of any one of embodiments 1-5, wherein the ratio of CBD to THC is about 100:1.

Embodiment 14: The pharmaceutical composition of any one of embodiments 1-4, wherein the ratio of CBD to THC is about 125:1.

Embodiment 15: The pharmaceutical composition of any one of embodiments 1-4, wherein the ratio of CBD to THC is about 150:1.

Embodiment 16: The pharmaceutical composition of any one of embodiments 1-4, wherein the ratio of CBD to THC is about 200:1.

Embodiment 17: The pharmaceutical composition of any one of embodiments 1-16, wherein the composition comprises from about 0.1% to about 99% of THC.

Embodiment 18: The pharmaceutical composition of any one of embodiments 1-17, wherein the composition comprises from about 1% to about 99.9% of CBD.

Embodiment 19: The pharmaceutical composition of any one of embodiments 1-8, further comprising a terpenoid.

Embodiment 20: The pharmaceutical composition of embodiment 19, wherein the terpenoid comprises myrcene, β-caryophyllene, limonene, α-terpineol, linalool, α-phellandrene, α-pinene, β-pinene, γ-terpinene, nerolidol, phytol, caryophyllene oxide, or α-humule.

Embodiment 21: The pharmaceutical composition of any one of embodiments 1-20, further comprising an analgesic agent.

Embodiment 22: The pharmaceutical composition of embodiment 21, wherein the analgesic agent comprises paracetamol, acetaminophen, a non-steroidal anti-inflammatory drug, a 5-HT (serotonin) receptor agonist, a calcitonin gene related peptide (CGRP) receptor antagonist, an N-methyl D-aspartate (NMDA) inhibitor, a glutamate receptor antagonist, a Substance P inhibitor, a GABA inhibitor, FAAH inhibitor, a Nerve Growth Factor (NGF) inhibitor, a Brain-Derived Neurotrophic Factor (BDNF) inhibitor, an extracellular signal-regulated kinase (ERK) antagonist, or a NO inhibitor.

Embodiment 23: The pharmaceutical composition of any one of embodiments 1-22, further comprising at least one minor cannabinoid.

Embodiment 24: The pharmaceutical composition of embodiment 23, wherein the at least one minor cannabinoid is selected from the group consisting of CBG, CBGA, CBN, CBNA, THCV, THCA, CBC, CBCA, CBDV, and D8-THC.

Embodiment 25: The pharmaceutical composition of embodiment 23 or 24, wherein the at least one minor cannabinoid is at least 90 wt. % pure.

Embodiment 26: The pharmaceutical composition of any one of embodiments 1-25, further comprising a pharmaceutically acceptable excipient.

Embodiment 27: The pharmaceutical composition of embodiment 26, wherein the pharmaceutically acceptable excipient comprises a diluent, a binder, a filler, a buffering agent, a pH modifying agent, a disintegrant, a dispersant, a preservative, a lubricant, a taste-masking agent, a flavoring agent, or a coloring agent.

Embodiment 28: The pharmaceutical composition of any one of embodiments 1-27, wherein the pharmaceutical composition is suitable for administration via injection.

Embodiment 29: The pharmaceutical composition of any one of embodiments 1-3 or 17-28, wherein the ratio of CBD to THC is from about 50:1 to about 150:1.

Embodiment 30: The pharmaceutical composition of any one of embodiments 1-3 or 17-28, wherein the ratio of CBD to THC is from about 75:1 to about 125:1.

Embodiment 31: The pharmaceutical composition of any one of embodiments 1-30, wherein the pharmaceutical composition is suitable for sublingual administration.

Embodiment 32: The composition of embodiment 31, wherein the pharmaceutical composition is a liquid solution suitable for administration as a spray.

Embodiment 33: The pharmaceutical composition of any one of embodiments 1-33, wherein the pharmaceutical composition is suitable for administration by inhalation.

Embodiment 34: The pharmaceutical composition of any one of embodiments 1-34, wherein the composition comprises a maximum dose of CBD of no more than 500 mg.

Embodiment 35: The pharmaceutical composition of any one of embodiments 1-34, wherein the composition comprises a maximum dose of THC of no more than 5 mg.

Embodiment 36: A pharmaceutical composition suitable for injection comprising CBD and THC, wherein a weight ratio of CBD to THC is from about 30:1 to about 300:1.

Embodiment 37: The pharmaceutical composition of embodiment 36, wherein the THC and CBD are at least 90 wt. % pure.

Embodiment 38: The pharmaceutical composition of embodiment 36 or 37, wherein the ratio of CBD to THC is from about 30:1 to about 100:1.

Embodiment 39: The pharmaceutical composition of any one of embodiments 36-38, wherein the ratio of CBD to THC is about 50:1.

Embodiment 40: The pharmaceutical composition of any one of embodiments 36-38, wherein the ratio of CBD to THC is about 60:1.

Embodiment 41: The pharmaceutical composition of any one of embodiments 36-38, wherein the ratio of CBD to THC is about 70:1.

Embodiment 42: The pharmaceutical composition of any one of embodiments 36-38, wherein the ratio of CBD to THC is about 80:1.

Embodiment 43: The pharmaceutical composition of any one of embodiments 36-38, wherein the ratio of CBD to THC is about 90:1.

Embodiment 44: The pharmaceutical composition of any one of embodiments 36-38, wherein the ratio of CBD to THC is about 100:1.

Embodiment 45: The pharmaceutical composition of any one of embodiments 36-37, wherein the ratio of CBD to THC is about 125:1.

Embodiment 46: The pharmaceutical composition of any one of embodiments 36-37, wherein the ratio of CBD to THC is about 150:1.

Embodiment 47: The pharmaceutical composition of any one of embodiments 36-37, wherein the ratio of CBD to THC is about 200:1.

Embodiment 48: The pharmaceutical composition of any one of embodiments 36-47, wherein the composition comprises from about 0.1% to about 99% of THC.

Embodiment 49: The pharmaceutical composition of any one of embodiments 36-48, wherein the composition comprises from about 1% to about 99.9% of CBD.

Embodiment 50: The pharmaceutical composition of any one of embodiments 36-49, further comprising a terpenoid.

Embodiment 51: The pharmaceutical composition of embodiment 50, wherein the terpenoid comprises myrcene, β-caryophyllene, limonene, α-terpineol, linalool, α-phellandrene, α-pinene, β-pinene, γ-terpinene, nerolidol, phytol, caryophyllene oxide, or α-humule.

Embodiment 52: The pharmaceutical composition of any one of embodiments 36-51, further comprising an analgesic agent.

Embodiment 53: The pharmaceutical composition of embodiment 52, wherein the analgesic agent comprises paracetamol, acetaminophen, a non-steroidal anti-inflammatory drug, a 5-HT (serotonin) receptor agonist, a calcitonin gene related peptide (CGRP) receptor antagonist, an N-methyl D-aspartate (NMDA) inhibitor, a glutamate receptor antagonist, a Substance P inhibitor, a GABA inhibitor, FAAH inhibitor, a Nerve Growth Factor (NGF) inhibitor, a Brain-Derived Neurotrophic Factor (BDNF) inhibitor, an extracellular signal-regulated kinase (ERK) antagonist, or a NO inhibitor.

Embodiment 54: The pharmaceutical composition of any one of embodiments 36-38, further comprising at least one minor cannabinoid.

Embodiment 55: The pharmaceutical composition of embodiment 54, wherein the at least one minor cannabinoid is selected from the group consisting of CBG, CBGA, CBN, CBNA, THCV, THCA, CBC, CBCA, CBDV, and D8-THC.

Embodiment 56: The pharmaceutical composition of embodiment 54 or 55, wherein the at least one minor cannabinoid is at least 90 wt. % pure.

Embodiment 57: The pharmaceutical composition of any one of embodiments 36-56, further comprising a pharmaceutically acceptable excipient.

Embodiment 58: The pharmaceutical composition of embodiment 57, wherein the pharmaceutically acceptable excipient comprises a carrier, a diluent, a binder, a filler, a buffering agent, a pH modifying agent, a disintegrant, a dispersant, a preservative, a lubricant, a taste-masking agent, a flavoring agent, or a coloring agent.

Embodiment 59: The pharmaceutical composition of embodiment 36, wherein the ratio of CBD to THC is from about 50:1 to about 150:1.

Embodiment 60: The pharmaceutical composition of embodiment 36, wherein the ratio of CBD to THC is from about 75:1 to about 125:1.

Embodiment 61: A pharmaceutical composition suitable for administration to a subject in need thereof comprising at least one minor cannabinoid, CBD, and THC.

Embodiment 62: The pharmaceutical composition of embodiment 61, wherein the at least one minor cannabinoid is selected from the group consisting of CBG, CBGA, CBN, CBNA, THCV, THCA, CBC, CBCA, CBDV, and D8-THC.

Embodiment 63: The pharmaceutical composition of embodiment 61 or 62, wherein the at least one minor cannabinoid is at least 90 wt. % pure.

Embodiment 64: The pharmaceutical composition of any one of embodiments 61-63, wherein a weight ratio of CBD to THC is from about 10:1 to about 300:1.

Embodiment 65: The pharmaceutical composition of any one of embodiments 61-64, wherein the THC and CBD are at least 90 wt. % pure.

Embodiment 66: The pharmaceutical composition of any one of embodiments 61-65, further comprising a terpenoid.

Embodiment 67: The pharmaceutical composition of embodiment 66, wherein the terpenoid comprises myrcene, β-caryophyllene, limonene, α-terpineol, linalool, α-phellandrene, α-pinene, β-pinene, γ-terpinene, nerolidol, phytol, caryophyllene oxide, or α-humule.

Embodiment 68: The pharmaceutical composition of any one of embodiments 61-68, further comprising an analgesic agent.

Embodiment 69: The pharmaceutical composition of embodiment 68, wherein the analgesic agent comprises paracetamol, acetaminophen, a non-steroidal anti-inflammatory drug, a 5-HT (serotonin) receptor agonist, a calcitonin gene related peptide (CGRP) receptor antagonist, an N-methyl D-aspartate (NMDA) inhibitor, a glutamate receptor antagonist, a Substance P inhibitor, a GABA inhibitor, FAAH inhibitor, a Nerve Growth Factor (NGF) inhibitor, a Brain-Derived Neurotrophic Factor (BDNF) inhibitor, an extracellular signal-regulated kinase (ERK) antagonist, or a NO inhibitor.

Embodiment 70: The pharmaceutical composition of any one of embodiments 61-69, wherein the ratio of CBD to THC is from about 30:1 to about 100:1.

Embodiment 71: The pharmaceutical composition of any one of embodiments 61-70, wherein the ratio of CBD to THC is about 50:1.

Embodiment 72: The pharmaceutical composition of any one of embodiments 61-70, wherein the ratio of CBD to THC is about 60:1.

Embodiment 73: The pharmaceutical composition of any one of embodiments 61-70, wherein the ratio of CBD to THC is about 70:1.

Embodiment 74: The pharmaceutical composition of any one of embodiments 61-70, wherein the ratio of CBD to THC is about 80:1.

Embodiment 75: The pharmaceutical composition of any one of embodiments 61-70, wherein the ratio of CBD to THC is about 90:1.

Embodiment 76: The pharmaceutical composition of any one of embodiments 61-70, wherein the ratio of CBD to THC is about 100:1.

Embodiment 77: The pharmaceutical composition of any one of embodiments 61-69, wherein the ratio of CBD to THC is about 125:1.

Embodiment 78: The pharmaceutical composition of any one of embodiments 61-69, wherein the ratio of CBD to THC is about 150:1.

Embodiment 79: The pharmaceutical composition of any one of embodiments 61-69, wherein the ratio of CBD to THC is about 200:1.

Embodiment 80: The pharmaceutical composition of any one of embodiments 61-79, further comprising a pharmaceutically acceptable excipient.

Embodiment 81: The pharmaceutical composition of any one of embodiments 61-80, wherein the pharmaceutically acceptable excipient comprises a carrier, a diluent, a binder, a filler, a buffering agent, a pH modifying agent, a disintegrant, a dispersant, a preservative, a lubricant, a taste-masking agent, a flavoring agent, or a coloring agent.

Embodiment 82: The pharmaceutical composition of any one of embodiments 61-81, wherein the pharmaceutical composition is suitable for administration via injection.

Embodiment 83: The pharmaceutical composition of any one of embodiments 61-69 or 80-82, wherein the ratio of CBD to THC is from about 50:1 to about 150:1.

Embodiment 84: The pharmaceutical composition of any one of embodiments 61-69 or 80-82, wherein the ratio of CBD to THC is from about 75:1 to about 125:1.

Embodiment 85: A pharmaceutical composition suitable for injection comprising at least one minor cannabinoid, CBD, and THC.

Embodiment 86: The pharmaceutical composition of embodiment 85, wherein the at least one minor cannabinoid is selected from the group consisting of CBG, CBGA, CBN, CBNA, THCV, THCA, CBC, CBCA, CBDV, and D8-THC.

Embodiment 87: The pharmaceutical composition of embodiment 85 or 86, wherein the at least one minor cannabinoid is at least 90 wt. % pure.

Embodiment 88: The pharmaceutical composition of any one of embodiments 85-87, wherein the weight ratio of CBD to THC is from about 10:1 to about 300:1.

Embodiment 89: The pharmaceutical composition of any one of embodiments 85-88, wherein the THC and CBD are at least 90 wt. % pure.

Embodiment 90: The pharmaceutical composition of any one of embodiments 85-89, further comprising a terpenoid.

Embodiment 91: The pharmaceutical composition of embodiment 90, wherein the terpenoid comprises myrcene, β-caryophyllene, limonene, α-terpineol, linalool, α-phellandrene, α-pinene, β-pinene, v-terpinene, nerolidol, phytol, caryophyllene oxide, or α-humule.

Embodiment 92: The pharmaceutical composition of any one of embodiments 85-91, further comprising an analgesic agent.

Embodiment 93: The pharmaceutical composition of embodiment 92, wherein the analgesic agent comprises paracetamol, acetaminophen, a non-steroidal anti-inflammatory drug, a 5-HT (serotonin) receptor agonist, a calcitonin gene related peptide (CGRP) receptor antagonist, an N-methyl D-aspartate (NMDA) inhibitor, a glutamate receptor antagonist, a Substance P inhibitor, a GABA inhibitor, FAAH inhibitor, a Nerve Growth Factor (NGF) inhibitor, a Brain-Derived Neurotrophic Factor (BDNF) inhibitor, an extracellular signal-regulated kinase (ERK) antagonist, or a NO inhibitor.

Embodiment 94: The pharmaceutical composition of any one of embodiments 85-93, wherein the ratio of CBD to THC is from about 30:1 to about 100:1.

Embodiment 95: The pharmaceutical composition of any one of embodiments 85-94, wherein the ratio of CBD to THC is about 50:1.

Embodiment 96: The pharmaceutical composition of any one of embodiments 85-94, wherein the ratio of CBD to THC is about 60:1.

Embodiment 97: The pharmaceutical composition of any one of embodiments 85-94, wherein the ratio of CBD to THC is about 70:1.

Embodiment 98: The pharmaceutical composition of any one of embodiments 85-94, wherein the ratio of CBD to THC is about 80:1.

Embodiment 99: The pharmaceutical composition of any one of embodiments 85-94, wherein the ratio of CBD to THC is about 90:1.

Embodiment 100: The pharmaceutical composition of any one of embodiments 85-94, wherein the ratio of CBD to THC is about 100:1.

Embodiment 101: The pharmaceutical composition of any one of embodiments 85-93, wherein the ratio of CBD to THC is about 125:1.

Embodiment 102: The pharmaceutical composition of any one of embodiments 85-93, wherein the ratio of CBD to THC is about 150:1.

Embodiment 103: The pharmaceutical composition of any one of embodiments 85-93, wherein the ratio of CBD to THC is about 200:1.

Embodiment 104: The pharmaceutical composition of any one of embodiments 85-103, further comprising a pharmaceutically acceptable excipient.

Embodiment 105: The pharmaceutical composition of embodiment 104, wherein the pharmaceutically acceptable excipient comprises a carrier, a diluent, a binder, a filler, a buffering agent, a pH modifying agent, a disintegrant, a dispersant, a preservative, a lubricant, a taste-masking agent, a flavoring agent, or a coloring agent.

Embodiment 106: The pharmaceutical composition of embodiment 85, wherein the ratio of CBD to THC is from about 50:1 to about 150:1.

Embodiment 107: The pharmaceutical composition of embodiment 85, wherein the ratio of CBD to THC is from about 75:1 to about 125:1.

Embodiment 108: A method for treating acute migraine pain, the method comprising administering a pharmaceutical composition comprising CBD and THC to a subject in need thereof.

Embodiment 109: The method of embodiment 108, wherein the subject has chronic migraines.

Embodiment 110: The method of embodiment 108 or 109, wherein the subject has high frequency migraines.

Embodiment 111: The method of any one of embodiments 108-110, wherein the administering is accomplished via injection.

Embodiment 112: The method of any one of embodiments 108-111, wherein the treatment is effective within at least two hours after administration.

Embodiment 113: The method of any one of embodiments 108-112, wherein the acute migraine pain is measured by a pain scale.

Embodiment 114: The method of any one of embodiments 108-113, wherein at least one biomarker of central and peripheral sensitization is modulated as a result of the administration.

Embodiment 115: The method of embodiment 114, wherein the at least one biomarker is selected from the group consisting of NMDA, glutamate, CGRP, FAAH, Substance P, 5-HT, NO, GABA, NGF, serotonin, dopamine, AEA, and 2-AG.

Embodiment 116: The method of any one of embodiments 108-115, wherein the pharmaceutical composition comprises an amount of CBD effective to reduce unwanted side effects of THC.

Embodiment 117: The method of embodiment 116, wherein the unwanted side effects of THC comprise psychoactivity, psychosis, anxiety, paranoia, dizziness, confusion, cardiovascular risk, memory impairment, or combinations thereof.

Embodiment 118: The method of any one of embodiments 108-117, wherein a weight ratio of CBD to THC is from about 10:1 to about 300:1.

Embodiment 119: The method of any one of embodiments 108-118, wherein the weight ratio of CBD to THC is from about 30:1 to about 100:1.

Embodiment 120: The method of any one of embodiments 108-118, wherein the weight ratio of CBD to THC is about 30:1.

Embodiment 121: The method of any one of embodiments 108-118, wherein the weight ratio of CBD to THC is about 50:1.

Embodiment 122: The method of any one of embodiments 108-118, wherein the weight ratio of CBD to THC is about 75:1.

Embodiment 123: The method of any one of embodiments 108-118, wherein the weight ratio of CBD to THC is about 100:1.

Embodiment 124: The method of any one of embodiments 108-118, wherein the subject is human.

Embodiment 125: The method of any one of embodiments 108-118, wherein the pharmaceutical composition further comprises a terpenoid.

Embodiment 126: The method of embodiment 125, wherein the terpenoid comprises myrcene, β-caryophyllene, limonene, α-terpineol, linalool, α-phellandrene, α-pinene, β-pinene, γ-terpinene, nerolidol, phytol, caryophyllene oxide, or α-humule.

Embodiment 127: The method of any one of embodiments 108-126, wherein the pharmaceutical composition further comprises an analgesic agent.

Embodiment 128: The method of embodiment 127, wherein the analgesic agent comprises paracetamol, acetaminophen, a non-steroidal anti-inflammatory drug, a 5-HT (serotonin) receptor agonist, a calcitonin gene related peptide (CGRP) receptor antagonist, an N-methyl D-aspartate (NMDA) inhibitor, a glutamate receptor antagonist, a Substance P inhibitor, a GABA inhibitor, FAAH inhibitor, a Nerve Growth Factor (NGF) inhibitor, a Brain-Derived Neurotrophic Factor (BDNF) inhibitor, an extracellular signal-regulated kinase (ERK) antagonist, or a NO inhibitor.

Embodiment 129: The method of any one of embodiments 108-128, wherein the pharmaceutical composition further comprises a pharmaceutically acceptable excipient.

Embodiment 130: The method of any one of embodiments 108-129, wherein the pharmaceutically acceptable excipient comprises a carrier, a diluent, a binder, a filler, a buffering agent, a pH modifying agent, a disintegrant, a dispersant, a preservative, a lubricant, a taste-masking agent, a flavoring agent, or a coloring agent.

Embodiment 131: The method of any one of embodiments 108-130, wherein the THC and CBD are at least 90 wt. % pure.

Embodiment 132: The method of any one of embodiments 108-131, wherein the pharmaceutical composition further comprises at least one minor cannabinoid.

Embodiment 133: The method of embodiment 132, wherein the at least one minor cannabinoid is selected from the group consisting of CBG, CBGA, CBN, CBNA, THCV, THCA, CBC, CBCA, CBDV, and D8-THC.

Embodiment 134: The method of embodiment 132 or 133, wherein the at least one minor cannabinoid is at least 90 wt. % pure.

Embodiment 135: The method of embodiment 108, wherein the weight ratio of CBD to THC is from about 50:1 to about 150:1.

Embodiment 136: The method of embodiment 108, wherein the weight ratio of CBD to THC is from about 75:1 to about 125:1.

Embodiment 137: A method for treating acute migraine pain, the method comprising administering a pharmaceutical composition comprising at least one minor cannabinoid, CBD, and THC to a subject in need thereof.

Embodiment 138: The method of embodiment 137, wherein the subject has chronic migraines.

Embodiment 139: The method of embodiment 137 or 138, wherein the subject has high frequency migraines.

Embodiment 140: The method of any one of embodiments 137-139, wherein the administering is accomplished via injection.

Embodiment 141: The method of any one of embodiments 137-140, wherein a treatment is effective within at least two hours after administration.

Embodiment 142: The method of any one of embodiments 137-141, wherein the acute migraine pain is measured by a pain scale.

Embodiment 143: The method of any one of embodiments 137-142, wherein at least one biomarker of central and peripheral sensitization is modulated as a result of the administration.

Embodiment 144: The method of any one of embodiments 137-143, wherein the at least one biomarker is selected from the group consisting of NMDA, glutamate, CGRP, FAAH, Substance P, 5-HT, NO, GABA, NGF, serotonin, dopamine, AEA, and 2-AG.

Embodiment 145: The method of any one of embodiments 137-144, wherein the pharmaceutical composition comprises an amount of CBD effective to reduce unwanted side effects of THC.

Embodiment 146: The method of any one of embodiments 137-145, wherein the unwanted side effects of THC comprise psychoactivity, psychosis, anxiety, paranoia, dizziness, confusion, cardiovascular risk, memory impairment, or combinations thereof.

Embodiment 147: The method of any one of embodiments 137-146, wherein a weight ratio of CBD to THC is from about 10:1 to about 300:1.

Embodiment 148: The method of any one of embodiments 137-147, wherein the weight ratio of CBD to THC is from about 30:1 to about 100:1.

Embodiment 149: The method of any one of embodiments 137-148, the weight ratio of CBD to THC is about 30:1.

Embodiment 150: The method of any one of embodiments 137-148, the weight ratio of CBD to THC is about 50:1.

Embodiment 151: The method of any one of embodiments 137-148, the weight ratio of CBD to THC is about 75:1.

Embodiment 152: The method of any one of embodiments 137-148, the weight ratio of CBD to THC is about 100:1.

Embodiment 153: The method of any one of embodiments 137-152, wherein the subject is human.

Embodiment 154: The method of any one of embodiments 137-153, wherein the pharmaceutical composition further comprises a terpenoid.

Embodiment 155: The method of embodiment 154, wherein the terpenoid comprises myrcene, β-caryophyllene, limonene, α-terpineol, linalool, α-phellandrene, α-pinene, β-pinene, γ-terpinene, nerolidol, phytol, caryophyllene oxide, or α-humule.

Embodiment 156: The method of any one of embodiments 137-155, wherein the pharmaceutical composition further comprises an analgesic agent.

Embodiment 157: The method of embodiment 156, wherein the analgesic agent comprises paracetamol, acetaminophen, a non-steroidal anti-inflammatory drug, a 5-HT (serotonin) receptor agonist, a calcitonin gene related peptide (CGRP) receptor antagonist, an N-methyl D-aspartate (NMDA) inhibitor, a glutamate receptor antagonist, a Substance P inhibitor, a GABA inhibitor, FAAH inhibitor, a Nerve Growth Factor (NGF) inhibitor, a Brain-Derived Neurotrophic Factor (BDNF) inhibitor, an extracellular signal-regulated kinase (ERK) antagonist, or a NO inhibitor.

Embodiment 158: The method of any one of embodiments 137-157, wherein the pharmaceutical composition further comprises a pharmaceutically acceptable excipient.

Embodiment 159: The method of any one of embodiments 137-158, wherein the pharmaceutically acceptable excipient comprises a carrier, a diluent, a binder, a filler, a buffering agent, a pH modifying agent, a disintegrant, a dispersant, a preservative, a lubricant, a taste-masking agent, a flavoring agent, or a coloring agent.

Embodiment 160: The method of any one of embodiments 137-159, wherein the THC and CBD are at least 90 wt. % pure.

Embodiment 161: The method of any one of embodiments 137-160, wherein the at least one minor cannabinoid is selected from the group consisting of CBG, CBGA, CBN, CBNA, THCV, THCA, CBC, CBCA, CBDV, and D8-THC.

Embodiment 162: The method of any one of embodiments 137-161, wherein the at least one minor cannabinoid is at least 90 wt. % pure.

Embodiment 163: The method of any one of embodiments 137-162, wherein the weight ratio of CBD to THC is from about 50:1 to about 150:1.

Embodiment 164: The method of any one of embodiments 137-163, wherein the weight ratio of CBD to THC is from about 75:1 to about 125:1.

Embodiment 165: A method for reducing acute migraine pain, the method comprising administering a pharmaceutical composition comprising at least one minor cannabinoid, CBD, and THC to a subject in need thereof.

Embodiment 166: The method of embodiment 165, wherein the subject has chronic migraines.

Embodiment 167: The method of embodiment 165 or 166, wherein the subject has high frequency migraines.

Embodiment 168: The method of any one of embodiments 165-167, wherein the administering is accomplished via injection.

Embodiment 169: The method of any one of embodiments 165-168, wherein a treatment is effective within at least two hours after administration.

Embodiment 170: The method of any one of embodiments 165-169, wherein the acute migraine pain is measured by a pain scale.

Embodiment 171: The method of any one of embodiments 165-170, wherein at least one biomarker of central and peripheral sensitization is modulated as a result of the administration.

Embodiment 172: The method of embodiment 171, wherein the at least one biomarker is selected from the group consisting of NMDA, glutamate, CGRP, FAAH, Substance P, 5-HT, NO, GABA, NGF, serotonin, dopamine, AEA, and 2-AG.

Embodiment 173: The method of any one of embodiments 165-172, wherein the pharmaceutical composition comprises an amount of CBD effective to reduce unwanted side effects of THC.

Embodiment 174: The method of embodiment 173, wherein the unwanted side effects of THC comprise psychoactivity, psychosis, anxiety, paranoia, dizziness, confusion, cardiovascular risk, memory impairment, or combinations thereof.

Embodiment 175: The method of any one of embodiments 165-174, wherein a weight ratio of CBD to THC is from about 10:1 to about 300:1.

Embodiment 176: The method of any one of embodiments 165-175, wherein the weight ratio of CBD to THC is from about 30:1 to about 100:1.

Embodiment 177: The method of any one of embodiments 165-176, the weight ratio of CBD to THC is about 30:1.

Embodiment 178: The method of any one of embodiments 165-176, the weight ratio of CBD to THC is about 50:1.

Embodiment 179: The method of any one of embodiments 165-176, the weight ratio of CBD to THC is about 75:1.

Embodiment 180: The method of any one of embodiments 165-176, the weight ratio of CBD to THC is about 100:1.

Embodiment 181: The method of any one of embodiments 165-180, wherein the subject is human.

Embodiment 182: The method of any one of embodiments 165-181, wherein the pharmaceutical composition further comprises a terpenoid.

Embodiment 183: The method of embodiments 182, wherein the terpenoid comprises myrcene, β-caryophyllene, limonene, α-terpineol, linalool, α-phellandrene, α-pinene, β-pinene, γ-terpinene, nerolidol, phytol, caryophyllene oxide, or α-humule.

Embodiment 184: The method of any one of embodiments 165-183, wherein the pharmaceutical composition further comprises an analgesic agent.

Embodiment 185: The method of embodiment 184, wherein the analgesic agent comprises paracetamol, acetaminophen, a non-steroidal anti-inflammatory drug, a 5-HT (serotonin) receptor agonist, a calcitonin gene related peptide (CGRP) receptor antagonist, an N-methyl D-aspartate (NMDA) inhibitor, a glutamate receptor antagonist, a Substance P inhibitor, a GABA inhibitor, FAAH inhibitor, a Nerve Growth Factor (NGF) inhibitor, a Brain-Derived Neurotrophic Factor (BDNF) inhibitor, an extracellular signal-regulated kinase (ERK) antagonist, or a NO inhibitor.

Embodiment 186: The method of any one of embodiments 165-185, wherein the pharmaceutical composition further comprises a pharmaceutically acceptable excipient.

Embodiment 187: The method of embodiment 186, wherein the pharmaceutically acceptable excipient comprises a carrier, a diluent, a binder, a filler, a buffering agent, a pH modifying agent, a disintegrant, a dispersant, a preservative, a lubricant, a taste-masking agent, a flavoring agent, or a coloring agent.

Embodiment 188: The method of any one of embodiments 165-187, wherein the THC and CBD are at least 90 wt. % pure.

Embodiment 189: The method of any one of embodiments 165-188, wherein the pharmaceutical composition further comprises at least one minor cannabinoid.

Embodiment 190: The method of embodiment 189, wherein the at least one minor cannabinoid is selected from the group consisting of CBG, CBGA, CBN, CBNA, THCV, THCA, CBC, CBCA, CBDV, and D8-THC.

Embodiment 191: The method of embodiment 189 or 190, wherein the at least one minor cannabinoid is at least 90 wt. % pure.

Embodiment 192: The method of embodiment 165, wherein the weight ratio of CBD to THC is from about 50:1 to about 150:1.

Embodiment 193: The method of embodiment 165, wherein the weight ratio of CBD to THC is from about 75:1 to about 125:1.

Embodiment 194: A method for reducing acute migraine pain, the method comprising administering a pharmaceutical composition comprising at least one minor cannabinoid, CBD, and THC to a subject in need thereof.

Embodiment 195: The method of embodiment 194, wherein the subject has chronic migraines.

Embodiment 196: The method of embodiment 194 or 195, wherein the subject has high frequency migraines.

Embodiment 197: The method of any one of embodiments 194-196, wherein the administering is accomplished via injection.

Embodiment 198: The method of any one of embodiments 194-197, wherein the treatment is effective within at least two hours after administration.

Embodiment 199: The method of any one of embodiments 194-198, wherein the acute migraine pain is measured by a pain scale.

Embodiment 200: The method of any one of embodiments 194-199, wherein at least one biomarker of central and peripheral sensitization is modulated as a result of the administration.

Embodiment 201: The method of embodiment 200, wherein the at least one biomarker is selected from the group consisting of NMDA, glutamate, CGRP, FAAH, Substance P, 5-HT, NO, GABA, NGF, serotonin, dopamine, AEA, and 2-AG.

Embodiment 202: The method of any one of embodiments 194-201, wherein the pharmaceutical composition comprises an amount of CBD effective to reduce unwanted side effects of THC.

Embodiment 203: The method of embodiment 202, wherein the unwanted side effects of THC comprise psychoactivity, psychosis, anxiety, paranoia, dizziness, confusion, cardiovascular risk, memory impairment, or combinations thereof.

Embodiment 204: The method of any one of embodiments 194-203, wherein a weight ratio of CBD to THC is from about 10:1 to about 300:1.

Embodiment 205: The method of any one of embodiments 194-204, wherein the weight ratio of CBD to THC is from about 30:1 to about 100:1.

Embodiment 206: The method of any one of embodiments 194-205, the weight ratio of CBD to THC is about 30:1.

Embodiment 207: The method of any one of embodiments 194-205, the weight ratio of CBD to THC is about 50:1.

Embodiment 208: The method of any one of embodiments 194-205, the weight ratio of CBD to THC is about 75:1.

Embodiment 209: The method of any one of embodiments 194-205, the weight ratio of CBD to THC is about 100:1.

Embodiment 210: The method of claim any one of embodiments 194-209, wherein the subject is human.

Embodiment 211: The method of any one of embodiments 194-210, wherein the pharmaceutical composition further comprises a terpenoid.

Embodiment 212: The method of embodiment 211, wherein the terpenoid comprises myrcene, β-caryophyllene, limonene, α-terpineol, linalool, α-phellandrene, α-pinene, β-pinene, γ-terpinene, nerolidol, phytol, caryophyllene oxide, or α-humule.

Embodiment 213: The method of any one of embodiments 194-212, wherein the pharmaceutical composition further comprises an analgesic agent.

Embodiment 214: The method of embodiment 213, wherein the analgesic agent comprises paracetamol, acetaminophen, a non-steroidal anti-inflammatory drug, a 5-HT (serotonin) receptor agonist, a calcitonin gene related peptide (CGRP) receptor antagonist, an N-methyl D-aspartate (NMDA) inhibitor, a glutamate receptor antagonist, a Substance P inhibitor, a GABA inhibitor, FAAH inhibitor, a Nerve Growth Factor (NGF) inhibitor, a Brain-Derived Neurotrophic Factor (BDNF) inhibitor, an extracellular signal-regulated kinase (ERK) antagonist, or a NO inhibitor.

Embodiment 215: The method of any one of embodiments 194-214, wherein the pharmaceutical composition further comprises a pharmaceutically acceptable excipient.

Embodiment 216: The method of embodiment 215, wherein the pharmaceutically acceptable excipient comprises a carrier, a diluent, a binder, a filler, a buffering agent, a pH modifying agent, a disintegrant, a dispersant, a preservative, a lubricant, a taste-masking agent, a flavoring agent, or a coloring agent.

Embodiment 217: The method of any one of embodiments 194-216, wherein the THC and CBD are at least 90 wt. % pure.

Embodiment 218: The method of any one of embodiments 194-217, wherein the at least one minor cannabinoid is selected from the group consisting of CBG, CBGA, CBN, CBNA, THCV, THCA, CBC, CBCA, CBDV, and D8-THC.

Embodiment 219: The method of any one of embodiments 194-218, wherein the at least one minor cannabinoid is at least 90 wt. % pure.

Embodiment 220: The method of embodiment 194, wherein the weight ratio of CBD to THC is from about 50:1 to about 150:1.

Embodiment 221: The method of embodiment 194, wherein the weight ratio of CBD to THC is from about 75:1 to about 125:1.

Embodiment 222: A method for reducing spontaneous pain, the method comprising administering a pharmaceutical composition comprising at least one minor cannabinoid, CBD, and THC to a subject in need thereof.

Embodiment 223: The method of embodiment 222, wherein the spontaneous pain is measured by a facial grimace.

Embodiment 224: The method of embodiment 222 or 223, wherein the subject is a male.

Embodiment 225: The method of any one of embodiments 222-224, wherein the subject is a human.

Embodiment 226: The method of any one of embodiments 222-225, wherein the weight ratio of CBD to THC is at least about 50:1.

Embodiment 227: The method of any one of embodiments 222-225, wherein the ratio of CBD to THC is at least about 75:1.

Embodiment 228: The method of claim 222, wherein the ratio of CBD to THC is at least about 100:1.

Embodiment 229: The method of any one of embodiments 222-225, wherein the ratio of CBD to THC is from about 50:1 to about 300:1.

Embodiment 230: The method of any one of embodiments 222-225, wherein the ratio of CBD to THC is from about 50:1 to about 200:1.

Embodiment 231: The method of any one of embodiments 222-225, wherein the ratio of CBD to THC is from about 50:1 to about 150:1.

Embodiment 232: The method of any one of embodiments 222-225, wherein the ratio of CBD to THC is from about 75:1 to about 125:1.

Embodiment 233: The method of any one of embodiments 222-232, wherein the administering is accomplished via injection.

Embodiment 234: The method of any one of embodiments 222-233, wherein the treatment is effective within at least two hours after administration.

Embodiment 235: The method of any one of embodiments 222-234, wherein the pharmaceutical composition further comprises a terpenoid.

Embodiment 236: The method of embodiment 235, wherein the terpenoid comprises myrcene, β-caryophyllene, limonene, α-terpineol, linalool, α-phellandrene, α-pinene, β-pinene, γ-terpinene, nerolidol, phytol, caryophyllene oxide, or α-humule.

Embodiment 237: The method of any one of embodiments 222-236, wherein the pharmaceutical composition further comprises an analgesic agent.

Embodiment 238: The method of embodiment 237, wherein the analgesic agent comprises paracetamol, acetaminophen, a non-steroidal anti-inflammatory drug, a 5-HT (serotonin) receptor agonist, a calcitonin gene related peptide (CGRP) receptor antagonist, an N-methyl D-aspartate (NMDA) inhibitor, a glutamate receptor antagonist, a Substance P inhibitor, a GABA inhibitor, FAAH inhibitor, a Nerve Growth Factor (NGF) inhibitor, a Brain-Derived Neurotrophic Factor (BDNF) inhibitor, an extracellular signal-regulated kinase (ERK) antagonist, or a NO inhibitor.

Embodiment 239: The method of any one of embodiments 222-238, wherein the pharmaceutical composition further comprises a pharmaceutically acceptable excipient.

Embodiment 240: The method of embodiment 239, wherein the pharmaceutically acceptable excipient comprises a carrier, a diluent, a binder, a filler, a buffering agent, a pH modifying agent, a disintegrant, a dispersant, a preservative, a lubricant, a taste-masking agent, a flavoring agent, or a coloring agent.

Embodiment 241: The method of any one of embodiments 222-240, wherein the THC and CBD are at least 90 wt. % pure.

Embodiment 242: The method of any one of embodiments 222-241, wherein the at least one minor cannabinoid is selected from the group consisting of CBG, CBGA, CBN, CBNA, THCV, THCA, CBC, CBCA, CDC, and D8-THC.

Embodiment 243: The method of any one of embodiments 222-242, wherein the at least one minor cannabinoid is at least 90 wt. % pure.

Embodiment 244: The method of any one of embodiments 222-225, wherein the weight ratio of CBD to THC is from about 50:1 to about 150:1.

Embodiment 245: The method of any one of embodiments 222-225, wherein the weight ratio of CBD to THC is from about 75:1 to about 125:1.

Embodiment 246: A method for treating hemiplegic migraine comprising administering a pharmaceutical composition comprising at least one minor cannabinoid, CBD, and THC to a subject in need thereof.

Embodiment 247: The method of embodiment 246, wherein the subject is a male.

Embodiment 248: The method of embodiment 246 or 247, wherein the subject is a human.

Embodiment 249: The method of any one of embodiments 246-248, wherein the administering is accomplished via injection.

Embodiment 250: The method of any one of embodiments 246-249, wherein the treatment is effective within at least two hours after administration.

Embodiment 251: The method of any one of embodiments 246-250, wherein the pharmaceutical composition further comprises a terpenoid.

Embodiment 252: The method of embodiment 251, wherein the terpenoid comprises myrcene, β-caryophyllene, limonene, α-terpineol, linalool, α-phellandrene, α-pinene, β-pinene, γ-terpinene, nerolidol, phytol, caryophyllene oxide, or α-humule.

Embodiment 253: The method of any one of embodiments 246-252, wherein the pharmaceutical composition further comprises an analgesic agent.

Embodiment 254: The method of embodiment 253, wherein the analgesic agent comprises paracetamol, acetaminophen, a non-steroidal anti-inflammatory drug, a 5-HT (serotonin) receptor agonist, a calcitonin gene related peptide (CGRP) receptor antagonist, an N-methyl D-aspartate (NMDA) inhibitor, a glutamate receptor antagonist, a Substance P inhibitor, a GABA inhibitor, FAAH inhibitor, a Nerve Growth Factor (NGF) inhibitor, a Brain-Derived Neurotrophic Factor (BDNF) inhibitor, an extracellular signal-regulated kinase (ERK) antagonist, or a NO inhibitor.

Embodiment 255: The method of any one of embodiments 246-254, wherein the pharmaceutical composition further comprises a pharmaceutically acceptable excipient.

Embodiment 256: The method of embodiment 255, wherein the pharmaceutically acceptable excipient comprises a carrier, a diluent, a binder, a filler, a buffering agent, a pH modifying agent, a disintegrant, a dispersant, a preservative, a lubricant, a taste-masking agent, a flavoring agent, or a coloring agent.

Embodiment 257: The method of any one of embodiments 246-256, wherein the THC and CBD are at least 90 wt. % pure.

Embodiment 258: The method of any one of embodiments 246-257, wherein the pharmaceutical composition further comprises at least one minor cannabinoid.

Embodiment 259: The method of embodiment 258, wherein the at least one minor cannabinoid is selected from the group consisting of CBG, CBGA, CBN, CBNA, THCV, THCA, CBC, CBCA, CBDV, and D8-THC.

Embodiment 260: The method of embodiment 258 or 259, wherein the at least one minor cannabinoid is at least 90 wt. % pure.

Embodiment 261: The method of embodiment 246, wherein a weight ratio of CBD to THC is from about 50:1 to about 150:1.

Embodiment 262: The method of embodiment 246, wherein the weight ratio of CBD to THC is from about 75:1 to about 125:1.

Embodiment 263: The method of any one of embodiments 246-262, wherein the hemiplegic migraine is familial hemiplegic migraine.

Embodiment 264: The method of any one of embodiments 246-263, wherein the hemiplegic migraine is sporadic hemiplegic migraine.

Embodiment 265: The method of any one of embodiments 246-264, wherein the pharmaceutical composition is administered upon a first symptom onset of acute attacks of hemiplegic migraine.

Embodiment 266: The method of any one of embodiments 246-265, wherein the pharmaceutical composition is administered within 5 minutes of the first symptom onset of acute attacks of hemiplegic migraine.

Embodiment 267: The method of any one of embodiments 246-266, wherein the pharmaceutical composition is administered within 10 minutes of the first symptom onset of acute attacks of hemiplegic migraine.

Embodiment 268: The method of any one of embodiments 246-267, wherein the pharmaceutical composition is administered within 30 minutes of the first symptom onset of acute attacks of hemiplegic migraine.

Embodiment 269: The method of any one of embodiments 246-268, wherein the pharmaceutical composition is administered within 1 hour of the first symptom onset of acute attacks of hemiplegic migraine.

Embodiment 270: The method of any one of embodiments 246-269, wherein the pharmaceutical composition is administered within 2 hours of the first symptom onset of acute attacks of hemiplegic migraine.

Embodiment 271: The method of any one of embodiments 246-270, wherein the pharmaceutical composition is administered within 4 hours of the first symptom onset of acute attacks of hemiplegic migraine.

Embodiment 272: The method of any one of embodiments 246-271, further comprising administering a second dose of the pharmaceutical composition 90 minutes or more after the first administration.

Embodiment 273: The method of embodiments 272, wherein no more than 2 administrations are performed within 24 hours.

Embodiment 274: The method of any one of embodiments 246-273, wherein the subject is free of headache pain within 15 minutes, 30 minutes, 1 hour, 4 hours, 24 hours, or 48 hours of the administration.

Embodiment 275: The method of any one of embodiments 246-274, wherein the subject experiences reduced headache pain within 15 minutes, 30 minutes, 1 hour, 4 hours, 24 hours, or 48 hours of the administration.

Embodiment 276: The method of any one of embodiments 246-275, wherein the subject experiences reduced phonophobia within 15 minutes, 30 minutes, 1 hour, 4 hours, 24 hours, or 48 hours of the administration.

Embodiment 277: The method of any one of embodiments 246-276, wherein the subject experiences reduced nausea within 15 minutes, 30 minutes, 1 hour, 4 hours, 24 hours, or 48 hours of the administration.

Embodiment 278: The method of any one of embodiments 246-277, wherein the subject has no headache pain 2 hours or more after the administration.

Embodiment 279: The method of any one of embodiments 246-278, wherein the subject is free of hemiplegia within 15 minutes, 30 minutes, 1 hour, 4 hours, 24 hours, or 48 hours of the administration.

Embodiment 280: The method of any one of embodiments 246-279, wherein the subject experiences reduced arm hemiplegia within 15 minutes, 30 minutes, 1 hour, 4 hours, 24 hours, or 48 hours of the administration.

Embodiment 281: The method of any one of embodiments 246-280, wherein the subject experiences reduced leg hemiplegia within 15 minutes, 30 minutes, 1 hour, 4 hours, 24 hours, or 48 hours of the administration.

Embodiment 282: A method for treating familial hemiplegic migraine comprising administering a pharmaceutical composition comprising at least one minor cannabinoid, CBD, and THC to a subject in need thereof.

Embodiment 283: The method of embodiment 282, wherein the subject is a male.

Embodiment 284: The method of embodiment 282 or 283, wherein the subject is a human.

Embodiment 285: The method of any one of embodiments 282-284, wherein the administering is accomplished via injection.

Embodiment 286: The method of any one of embodiments 282-285, wherein the treatment is effective within at least two hours after administration.

Embodiment 287: The method of any one of embodiments 282-286, wherein the pharmaceutical composition further comprises a terpenoid.

Embodiment 288: The method of embodiment 287, wherein the terpenoid comprises myrcene, β-caryophyllene, limonene, α-terpineol, linalool, α-phellandrene, α-pinene, β-pinene, γ-terpinene, nerolidol, phytol, caryophyllene oxide, or α-humule.

Embodiment 289: The method of any one of embodiments 282-288, wherein the pharmaceutical composition further comprises an analgesic agent.

Embodiment 290: The method of embodiment 289, wherein the analgesic agent comprises paracetamol, acetaminophen, a non-steroidal anti-inflammatory drug, a 5-HT (serotonin) receptor agonist, a calcitonin gene related peptide (CGRP) receptor antagonist, an N-methyl D-aspartate (NMDA) inhibitor, a glutamate receptor antagonist, a Substance P inhibitor, a GABA inhibitor, FAAH inhibitor, a Nerve Growth Factor (NGF) inhibitor, a Brain-Derived Neurotrophic Factor (BDNF) inhibitor, an extracellular signal-regulated kinase (ERK) antagonist, or a NO inhibitor.

Embodiment 291: The method of any one of embodiments 282-291, wherein the pharmaceutical composition further comprises a pharmaceutically acceptable excipient.

Embodiment 292: The method of embodiment 291, wherein the pharmaceutically acceptable excipient comprises a carrier, a diluent, a binder, a filler, a buffering agent, a pH modifying agent, a disintegrant, a dispersant, a preservative, a lubricant, a taste-masking agent, a flavoring agent, or a coloring agent.

Embodiment 293: The method of any one of embodiments 282-292, wherein the THC and CBD are at least 90 wt. % pure.

Embodiment 294: The method of any one of embodiments 282-293, wherein the pharmaceutical composition further comprises at least one minor cannabinoid.

Embodiment 295: The method of embodiment 294, wherein the at least one minor cannabinoid is selected from the group consisting of CBG, CBGA, CBN, CBNA, THCV, THCA, CBC, CBCA, CBDV, and D8-THC.

Embodiment 296: The method of embodiment 294 or 295, wherein the at least one minor cannabinoid is at least 90 wt. % pure.

Embodiment 297: The method of any one of embodiments 282-296, wherein a weight ratio of CBD to THC is from about 50:1 to about 150:1.

Embodiment 298: The method of any one of embodiments 282-297, wherein the weight ratio of CBD to THC is from about 75:1 to about 125:1.

Embodiment 299: The method of any one of embodiments 282-298, wherein the familial hemiplegic migraine is familial hemiplegic migraine type 1.

Embodiment 300: The method of any one of embodiments 282-298, wherein the familial hemiplegic migraine is familial hemiplegic migraine type 2.

Embodiment 301: The method of any one of embodiments 282-298, wherein the familial hemiplegic migraine is familial hemiplegic migraine type 3.

Embodiment 302: The method of any one of embodiments 282-301, wherein the pharmaceutical composition is administered upon a first symptom onset of acute attacks of familial hemiplegic migraine.

Embodiment 303: The method of any one of embodiments 282-302, wherein the pharmaceutical composition is administered within 5 minutes of the first symptom onset of acute attacks of familial hemiplegic migraine.

Embodiment 304: The method of any one of embodiments 282-303, wherein the pharmaceutical composition is administered within 10 minutes of the first symptom onset of acute attacks of familial hemiplegic migraine.

Embodiment 305: The method of any one of embodiments 282-304, wherein the pharmaceutical composition is administered within 30 minutes of the first symptom onset of acute attacks of familial hemiplegic migraine.

Embodiment 306: The method of any one of embodiments 282-305, wherein the pharmaceutical composition is administered within 1 hour of the first symptom onset of acute attacks of familial hemiplegic migraine.

Embodiment 307: The method of any one of embodiments 282-306, wherein the pharmaceutical composition is administered within 2 hours of the first symptom onset of acute attacks of familial hemiplegic migraine.

Embodiment 308: The method of any one of embodiments 282-307, wherein the pharmaceutical composition is administered within 4 hours of the first symptom onset of acute attacks of familial hemiplegic migraine.

Embodiment 309: The method of any one of embodiments 282-308, further comprising administering a second dose of the pharmaceutical composition 90 minutes or more after the first administration.

Embodiment 310: The method of any one of embodiments 282-309, wherein no more than 2 administrations are performed within 24 hours.

Embodiment 311: The method of any one of embodiments 282-310, wherein the subject is free of headache pain within 15 minutes, 30 minutes, 1 hour, 4 hours, 24 hours, or 48 hours of the administration.

Embodiment 312: The method of any one of embodiments 282-311, wherein the subject experiences reduced headache pain within 15 minutes, 30 minutes, 1 hour, 4 hours, 24 hours, or 48 hours of the administration.

Embodiment 313: The method of any one of embodiments 282-312, wherein the subject experiences reduced photophobia within 15 minutes, 30 minutes, 1 hour, 4 hours, 24 hours, or 48 hours of the administration.

Embodiment 314: The method of any one of embodiments 282-313, wherein the subject experiences reduced phonophobia within 15 minutes, 30 minutes, 1 hour, 4 hours, 24 hours, or 48 hours of the administration.

Embodiment 315: The method of any one of embodiments 282-314, wherein the subject experiences reduced nausea within 15 minutes, 30 minutes, 1 hour, 4 hours, 24 hours, or 48 hours of the administration.

Embodiment 316: The method of any one of embodiments 282-315, wherein the subject has no headache pain 2 hours or more after the administration.

Embodiment 317: The method of any one of embodiments 282-316, wherein the subject is free of hemiplegia within 15 minutes, 30 minutes, 1 hour, 4 hours, 24 hours, or 48 hours of the administration.

Embodiment 318: The method of any one of embodiments 282-317, wherein the subject experiences reduced arm hemiplegia within 15 minutes, 30 minutes, 1 hour, 4 hours, 24 hours, or 48 hours of the administration.

Embodiment 319: The method of any one of embodiments 282-318, wherein the subject experiences reduced leg hemiplegia within 15 minutes, 30 minutes, 1 hour, 4 hours, 24 hours, or 48 hours of the administration.

Embodiment 320: A method for treating sporadic hemiplegic migraine comprising administering a pharmaceutical composition comprising at least one minor cannabinoid, CBD, and THC to a subject in need thereof.

Embodiment 321: The method of embodiment 320, wherein the subject is a male.

Embodiment 322: The method of embodiment 320 or 321, wherein the subject is a human.

Embodiment 323: The method of any one of embodiments 320-322, wherein the administering is accomplished via injection.

Embodiment 324: The method of any one of embodiments 320-323, wherein the treatment is effective within at least two hours after administration.

Embodiment 325: The method of any one of embodiments 320-324, wherein the pharmaceutical composition further comprises a terpenoid.

Embodiment 326: The method of embodiment 325, wherein the terpenoid comprises myrcene, β-caryophyllene, limonene, α-terpineol, linalool, α-phellandrene, α-pinene, β-pinene, γ-terpinene, nerolidol, phytol, caryophyllene oxide, or α-humule.

Embodiment 327: The method of any one of embodiments 320-326, wherein the pharmaceutical composition further comprises an analgesic agent.

Embodiment 328: The method of embodiment 327, wherein the analgesic agent comprises paracetamol, acetaminophen, a non-steroidal anti-inflammatory drug, a 5-HT (serotonin) receptor agonist, a calcitonin gene related peptide (CGRP) receptor antagonist, an N-methyl D-aspartate (NMDA) inhibitor, a glutamate receptor antagonist, a Substance P inhibitor, a GABA inhibitor, FAAH inhibitor, a Nerve Growth Factor (NGF) inhibitor, a Brain-Derived Neurotrophic Factor (BDNF) inhibitor, an extracellular signal-regulated kinase (ERK) antagonist, or a NO inhibitor.

Embodiment 329: The method of any one of embodiments 320-328, wherein the pharmaceutical composition further comprises a pharmaceutically acceptable excipient.

Embodiment 330: The method of embodiment 329, wherein the pharmaceutically acceptable excipient comprises a carrier, a diluent, a binder, a filler, a buffering agent, a pH modifying agent, a disintegrant, a dispersant, a preservative, a lubricant, a taste-masking agent, a flavoring agent, or a coloring agent.

Embodiment 331: The method of any one of embodiments 320-330, wherein the THC and CBD are at least 90 wt. % pure.

Embodiment 332: The method of any one of embodiments 320-331, wherein the pharmaceutical composition further comprises at least one minor cannabinoid.

Embodiment 333: The method of embodiment 332, wherein the at least one minor cannabinoid is selected from the group consisting of CBG, CBGA, CBN, CBNA, THCV, THCA, CBC, CBCA, CBDV, and D8-THC.

Embodiment 334: The method of embodiment 332 or 333, wherein the at least one minor cannabinoid is at least 90 wt. % pure.

Embodiment 335: The method of any one of embodiments 320-334, wherein the weight ratio of CBD to THC is from about 50:1 to about 150:1.

Embodiment 336: The method of any one of embodiments 320-335, wherein the weight ratio of CBD to THC is from about 75:1 to about 125:1.

Embodiment 337: The method of any one of embodiments 320-336, wherein the pharmaceutical composition is administered upon first symptom onset of acute attacks of sporadic hemiplegic migraine.

Embodiment 338: The method of any one of embodiments 320-337, wherein the pharmaceutical composition is administered within 5 minutes of the first symptom onset of acute attacks of sporadic hemiplegic migraine.

Embodiment 339: The method of any one of embodiments 320-338, wherein the pharmaceutical composition is administered within 10 minutes of the first symptom onset of acute attacks of sporadic hemiplegic migraine.

Embodiment 340: The method of any one of embodiments 320-339, wherein the pharmaceutical composition is administered within 30 minutes of the first symptom onset of acute attacks of sporadic hemiplegic migraine.

Embodiment 341: The method of any one of embodiments 320-340, wherein the pharmaceutical composition is administered within 1 hour of the first symptom onset of acute attacks of sporadic hemiplegic migraine.

Embodiment 342: The method of any one of embodiments 320-341, wherein the pharmaceutical composition is administered within 2 hours of the first symptom onset of acute attacks of sporadic hemiplegic migraine.

Embodiment 343: The method of any one of embodiments 320-342, wherein the pharmaceutical composition is administered within 4 hours of the first symptom onset of acute attacks of sporadic hemiplegic migraine.

Embodiment 344: The method of any one of embodiments 320-343, further comprising administering a second dose of the pharmaceutical composition 90 minutes or more after the first administration.

Embodiment 345: The method of embodiment 344, wherein no more than 2 administrations are performed within 24 hours.

Embodiment 346: The method of any one of embodiments 320-345, wherein the subject is free of headache pain within 15 minutes, 30 minutes, 1 hour, 4 hours, 24 hours, or 48 hours of the administration.

Embodiment 347: The method of any one of embodiments 320-346, wherein the subject experiences reduced headache pain within 15 minutes, 30 minutes, 1 hour, 4 hours, 24 hours, or 48 hours of the administration.

Embodiment 348: The method of any one of embodiments 320-347, wherein the subject experiences reduced photophobia within 15 minutes, 30 minutes, 1 hour, 4 hours, 24 hours, or 48 hours of the administration.

Embodiment 349: The method of any one of embodiments 320-348, wherein the subject experiences reduced phonophobia within 15 minutes, 30 minutes, 1 hour, 4 hours, 24 hours, or 48 hours of the administration.

Embodiment 350: The method of any one of embodiments 320-349, wherein the subject experiences reduced nausea within 15 minutes, 30 minutes, 1 hour, 4 hours, 24 hours, or 48 hours of the administration.

Embodiment 351: The method of any one of embodiments 320-350, wherein the subject has no headache pain 2 hours or more after the administration.

Embodiment 352: The method of any one of embodiments 320-351, wherein the subject is free of hemiplegia within 15 minutes, 30 minutes, 1 hour, 4 hours, 24 hours, or 48 hours of the administration.

Embodiment 353: The method of any one of embodiments 320-352, wherein the subject experiences reduced arm hemiplegia within 15 minutes, 30 minutes, 1 hour, 4 hours, 24 hours, or 48 hours of the administration.

Embodiment 354: The method of any one of embodiments 320-353, wherein the subject experiences reduced leg hemiplegia within 15 minutes, 30 minutes, 1 hour, 4 hours, 24 hours, or 48 hours of the administration.

Embodiment 355: A method for reducing the severity of hemiplegic migraine comprising administering a pharmaceutical composition comprising CBD and THC to a subject in need thereof.

Embodiment 356: A method for reducing the duration of hemiplegic migraine symptoms comprising administering a pharmaceutical composition comprising CBD and THC to a subject in need thereof.

Embodiment 357: A method for reducing the frequency of hemiplegic migraine attacks comprising administering a pharmaceutical composition comprising CBD and THC to a subject in need thereof.

Embodiment 358: A method for reducing the intensity of hemiplegic migraine symptoms comprising administering a pharmaceutical composition comprising CBD and THC to a subject in need thereof.

Embodiment 359: A method for preventing the onset of hemiplegic migraine symptoms comprising administering a pharmaceutical composition comprising CBD and THC to a subject in need thereof prior to the onset of hemiplegic migraine symptoms.

Embodiment 360: The method of embodiment 359, wherein the composition is administered up to one hour prior to the onset of hemiplegic migraine symptoms.

Embodiment 361: The method of embodiment 359, wherein the composition is administered up to 30 minutes prior to the onset of hemiplegic migraine symptoms.

Embodiment 362: The method of embodiment 359, wherein the composition is administered up to 15 minutes prior to the onset of hemiplegic migraine symptoms.

Embodiment 363: The method of embodiment 359, wherein the composition is administered up to 10 minutes prior to the onset of hemiplegic migraine symptoms.

Embodiment 364: The method of embodiment 359, wherein the composition is administered up to 5 minutes prior to the onset of hemiplegic migraine symptoms.

Embodiment 365: The method of any one of the previous embodiments, further comprising confirming the hemiplegic migraine genotype of the subject prior to administering the pharmaceutical composition.

Embodiment 366: A method for treating epilepsy comprising administering a pharmaceutical composition comprising CBD and THC to a subject in need thereof prior to the onset of hemiplegic migraine symptoms.

Embodiment 367: A method for preventing the onset of epilepsy symptoms comprising administering a pharmaceutical composition comprising CBD and THC to a subject in need thereof prior to the onset of hemiplegic migraine symptoms.

### Definitions

Unless defined otherwise, all technical and scientific terms used herein have the meaning commonly understood by a person skilled in the art to which this invention belongs.

Reference throughout this specification to “one embodiment,” “some embodiments”, “certain embodiments,” “one or more embodiments,” or “an embodiment” means that a particular feature, structure, material, or characteristic described in connection with the embodiment is included in at least one embodiment of the invention. Thus, the appearances of phrases containing the term “embodiment(s)” in various places throughout this specification are not necessarily referring to the same embodiment of the invention. Furthermore, the particular features, structures, materials, or characteristics may be combined in any suitable manner in one or more embodiments.

In the present disclosure, “%” refers to “weight % (wt. %)” or “mass %”, unless otherwise stated.

As used herein, the phrase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phrase “consisting essentially of” limits the scope of a claim to the specified elements or method steps, plus those that do not materially affect the basic and novel characteristic(s) of the claimed subject matter.

When introducing elements of the embodiments described herein, the articles “a”, “an”, “the”, and “said” are intended to mean that there are one or more of the elements.

The terms “comprises”, “comprising”, or any other variations thereof used in the disclosure, are intended to cover a non-exclusive inclusion, such that a device, apparatus, system, assembly, method that comprises a list of components or a series of steps that does not include only those components or steps but may include other components or steps not expressly listed or inherent to such apparatus, or assembly, or device. In other words, one or more elements or steps in a system or device or process proceeded by “comprises . . . a” or “comprising . . . of” does not, without more constraints, preclude the existence of other elements or additional elements or additional steps in the system, device, or process as the case may be. Besides, the use of “comprising”, “containing” or “including” also contemplates embodiments that “consist essentially of” or “consist of” the recited formulation and steps of preparation of the formulation.

Concentrations, amounts, and other numerical data may be expressed or presented herein in a range format. It is to be understood that such a range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 2 to about 50” should be interpreted to include not only the explicitly recited values of 2 to 50, but also include all individual values and sub-ranges within the indicated range. Thus, included in this numerical range are individual values such as 2, 2.4, 3, 3.7, 4, 5.5, 10, 10.1, 14, 15, 15.98, 20, 20.13, 23, 25.06, 30, 35.1, 38.0, 40, 44, 44.6, 45, 48, and sub-ranges such as from 1-3, from 2-4, from 5-10, from 5-20, from 5-25, from 5-30, from 5-35, from 5-40, from 5-50, from 2-10, from 2-20, from 2-30, from 2-40, from 2-50, etc. This same principle applies to ranges reciting only one numerical value as a minimum or a maximum. Furthermore, such an interpretation should apply regardless of the breadth of the range, or the characteristics being described.

As used herein, the term “about” is used to provide flexibility to a numerical range endpoint by providing that a given value may be “a little above” or “a little below” the endpoint. For example, the endpoint may be within 10%, 8%, 5%, 3%, 2%, or 1% of the listed value. Further, for the sake of convenience and brevity, a numerical range of “about 50 mg/mL to about 80 mg/mL” should also be understood to provide support for the range of “50 mg/mL to 80 mg/mL.” The endpoint may also be based on the variability allowed by an appropriate regulatory body, such as the FDA, USP, etc.

In this disclosure, the terms “including,” “containing,” and/or “having” are understood to mean “comprising” and are open ended terms.

As various changes could be made in the above-described methods without departing from the scope of the invention, it is intended that all matter contained in the above description and in the examples given below, shall be interpreted as illustrative and not in a limiting sense.

### EXAMPLES

### Example 1: Manufacturing of Dronabinol

The data presented in this example provides information of the manufacturing of dronabinol, analytical characterization of the intermediates of dronabinol, stability, and storage of dronabinol for use in the composition for treating hemiplegic migraines according to DMF 036220.

### Dronabinol General Information

International Nonproprietary Names (INN): Dronabinol

Other Non-proprietary names: A9-Tetrahydrocannabinol, D9-Tetrahydrocannabinol, D9-THC, (−)-trans-A9-tetrahydrocannabinol.

Chemical Names: 2-(6aR,10aR) 6a,7,8,10a-tetrahydro-6,6,9-trimethyl-3-pentyl-6H-dibenzo[b,d]pyran-l-ol.

CAS #: 1972 Aug. 3

Molecular Formula: C₂₁H₃₀O₂

Molecular Weight: 314.47 g/mol

DEA Controlled Substance Schedule: Schedule I.

Physical Properties/Appearance: Almost colorless to light yellow, oily fluid or resin-like mass, sometimes slightly purple at the surface

Solubility at 25° C.: Dronabinol is immiscible in water.

Solubility at 25° C. in organic solvents:

| | Solubility |
|---|---|
| Solvent | (mg/mL) |
| Ethanol | 100 |
| Acetone | 100 |

Hygroscopicity: Thermogravimetric analysis showed dronabinol to be non-hygroscopic with weight change of less than 0.1% at approximately 140° C.

Chirality/Specific Optical Rotation: Dronabinol (D9-THC) bears two stereogenic centers. Therefore, four stereoisomers are possible for this drug substance. With manufacturing controls in the process, the (6aR, 10aR)-isomer were obtained. Due to the manufacturing process, the stereochemistry was controlled due to the raw materials used, specifically menthadienol.

Polymorphism: Neat Dronabinol is an amorphous oil, not a crystalline solid at room temperature. Therefore, the presence of polymorphism does not apply.

### Manufacturing of Dronabinol Drug Substance, Overview of Preparation

The preparation of D9-THC (Dronabinol) is shown in the synthetic scheme in Scheme 1, shown below. Olivetol was reacted with menthadienol in presence of catalytic amounts of p-toluene sulfonic acid (pTSA) in toluene forming D8-tetrahydrocannabinol. The D8-tetrahydrocannabinol is esterified with naphthoyl chloride and triethylamine to form D8-tetrahydrocannabinol (THC) naphthoyl ester. This D8-THC naphthoylester is then precipitated and isolated from the solution by the addition of methanol. The D8-THC naphthoylester is further purified by recrystallization from ethanol. The purified D8-THC-naphthoylester is saponified using lithium hydroxide to form D8-THC. The D8-THC is converted to the D9-THC isomer by using the HCl adduct of D8-THC. The D9-THC adduct was esterified using naphthoyl chloride and triethylamine forming the D9-THC naphthoylester. The D9-THC naphthoylester was precipitated and further recrystallized. Saponification of the recrystallized naphthoylester using lithium hydroxide formed crude D9-THC (crude Dronabinol). The crude D9-THC (crude Dronabinol) is then purified by continuous distillation to obtain D9-THC (drug substance).

### Scheme 1: Manufacturing Scheme for Dronabinol

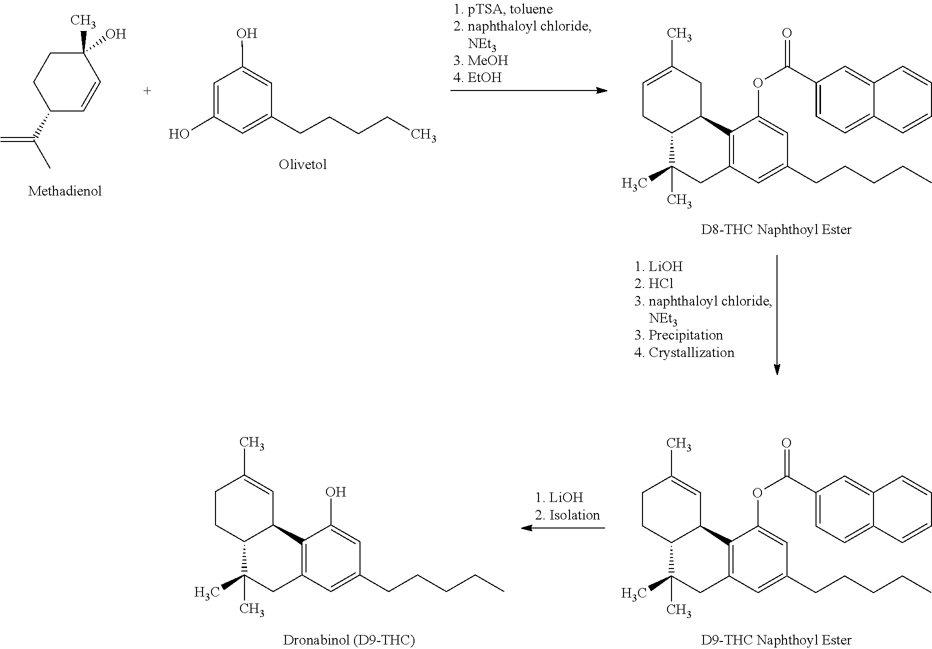

### Batch Analysis

Batches of clinical (developmental) dronabinol and validation batches were manufactured using the manufacturing process described above. General information regarding batch size is presented in Table 2 and Table 3.

TABLE 2
General Information on Dronabinol Clinical Batches

| Batch Number* | Date of Manufacture | Batch Size |
|---|---|---|
| 20-PUR-006 | December 2020 | 1.3 kg |
| 20-PUR-007 | December 2020 | 1.5 kg |
| 21-PUR-002 | January 2021 | 1.8 kg |

TABLE 3
General Information on Dronabinol Validation Batches

| Batch Number | Date of Manufacture | Batch Size |
|---|---|---|
| 21EP081-4 | May 2021 | 2.91 kg |
| 21EP082-4 | June 2021 | 3.16 kg |
| 21EP083-4 | June 2021 | 3.39 kg |

These batches were tested according to the tests and specifications for the drug substance provided below. The data demonstrates the consistency in the developmental and validation manufacturing batches; and compliance with the proposed drug substance specifications.

### Specifications of the Dronabinol Drug Substance

The specifications for dronabinol are summarized in Table 4. USP methods are used except for methods provided in this technical package.

TABLE 4
Specification for Dronabinol.

| | Acceptance | Test |
|---|---|---|
| Test Parameter | Criteria | Method |
| Appearance | Almost colorless | Method in |
| | to light yellow, | section 4.2.3 |
| | oily fluid or | |
| | resin-like mass, | |
| | sometimes slightly | |
| | purple at the | |
| | surface | |
| Identification by IR | Sample Spectra | USP <197> |
| | corresponds | |
| | with reference | |
| Identification by UPLC | Retention time | Method in |
| | corresponds with | section 4.2.1 |
| | reference | |
| Assay by UPLC (Area % | 95.0%- | Method in |
| Normalization) | 100.0% w/w | section 4.2.1 |

UPLC Purity, Specific Impurities

| | | |
|---|---|---|
| Cannabinol (CBN) | ≤1.0% w/w | Method in |
| D8-Tetrahydrocannabinol (D8-THC) | ≤2.0% w/w | section 4.2.1 |
| Each unspecified impurity | ≤1.0% w/w | |
| Sum of impurities except D8-THC | ≤3.0% w/w | |

Residual Solvents (ppm)

| | | |
|---|---|---|
| Methanol (MeOH) | ≤3000 ppm | Method in |
| Methyl tert-butyl ether (MTBE) | ≤5000 ppm | section 4.2.2 |
| Tetrahydrofuran (THF) | ≤720 ppm | |
| n-Heptane¹ | ≤5000 ppm | |

Microbiological Quantity²

| | | |
|---|---|---|
| TAMC | Max 10³ cfu/g | USP <61> |
| TYMC | Max 10² cfu/g | |

¹n-Heptane is only specified for clinical batches.
²Microbiological Quantity is only specified for commercial batches.

### Analytical Results for Clinical Batches of Dronabinol

The analytical data for the clinical (developmental) batches for dronabinol are shown below in Table 5.

TABLE 5
Analytical Data for Clinical Batches

| | Acceptance | 20-PUR- | 20-PUR- | 21-PUR- |
|---|---|---|---|---|
| Test Parameters | Criteria | 006 | 007 | 002 |
| Appearance | Almost | Complies | Complies | Complies |
| | colorless | | | |
| | to light | | | |
| | yellow, | | | |
| | oily fluid | | | |
| | or resin-like | | | |
| | mass, | | | |
| | sometimes | | | |
| | slightly | | | |
| | purple at | | | |
| | the surface | | | |
| Identification | Sample | Complies | Complies | Complies |
| by IR | Spectra | | | |
| | corresponds | | | |
| | with | | | |
| | reference | | | |
| Identification by | Retention time | Complies | Complies | Complies |
| UPLC | corresponds | | | |
| | with | | | |
| | reference | | | |
| Assay by UPLC | 95.0%-100.0% | 99.4% | 99.4% | 99.4% |
| (Area % | w/w | | | |
| Normalized) | | | | |

Specified Impurities

| | | | | |
|---|---|---|---|---|
| Cannabinol (CBN) | <1.0% w/w | 0.1% | <0.05% | <0.05% |
| | | | (ND) | |
| D8- | <2.0% w/w | 0.3% | 0.4% | 0.4% |
| Tetrahydro- | | | | |
| cannabinol | | | | |
| (D8-THC) | | | | |
| Each unspecified | <1.0% w/w | 0.1% | 0.1% | 0.1% |
| impurity | | | | |
| Sum of impurities | <3.0% w/w | 0.4% | 0.1% | 0.2% |
| except D8-THC | | | | |

Residual solvents (ppm)

| | | | | |
|---|---|---|---|---|
| Methanol (MeOH) | <3000 ppm | <300 ppm | <300 ppm | <300 ppm |
| Methyl tert-butyl | <5000 ppm | <500 ppm | <500 ppm | <500 ppm |
| ether (MTBE) | | | | |
| Tetrahydrofuran | <720 ppm | <72 ppm | 96 ppm | <72 ppm |
| (THF) | | | | |
| ¹n-Heptane | <5000 ppm | <500 ppm | 846 ppm | <500 ppm |

¹n-Heptane was only specified for clinical batches.
²Microbiological Quantity was only specified for commercial batches.

### Analytical Results for Validation Batches of Dronabinol

Table 6, shown below, shows the analytical data for the validation batches.

TABLE 6
Analytical data for Validation Batches.

| Test | Acceptance | 21EP081- | 21EP082- | 21EP083- |
|---|---|---|---|---|
| Parameters | Criteria | 4 | 4 | 4 |
| Appearance | Almost | Pass | Pass | Pass |
| | colorless | | | |
| | to light | | | |
| | yellow, oily | | | |
| | fluid or | | | |
| | resin-like | | | |
| | mass, | | | |
| | sometimes | | | |
| | slightly | | | |
| | purple at | | | |
| | the surface | | | |
| Identification | Sample | Pass | Pass | Pass |
| by IR | Spectra | | | |
| | corresponds | | | |
| | with | | | |
| | reference | | | |
| Identification | Retention time | Pass | Pass | Pass |
| by UPLC | corresponds | | | |
| | with | | | |
| | reference | | | |
| Assay by UPLC | 95.0%- | 99.5% | 99.5% | 99.5% |
| (Area % | 100.0% w/w | | | |
| Normalized) | | | | |

Specified Impurities

| | | | | |
|---|---|---|---|---|
| Cannabinol | <1.0% w/w | <0.1% | <0.1% | <0.1% |
| (CBN) | | | | |
| D8- | <2.0% w/w | 0.3% | 0.4% | 0.3% |
| Tetrahydro- | | | | |
| cannabinol | | | | |
| (D8-THC) | | | | |
| Each | <1.0% w/w | 0.1% | 0.1% | 0.1% |
| unspecified | | | | |
| impurity | | | | |
| Sum of | <3.0% w/w | 0.2% | 0.2% | 0.2% |
| impurities | | | | |
| except D8-THC | | | | |

Residual solvents (ppm)

| | | | | |
|---|---|---|---|---|
| Methanol | <3000 ppm | <300 ppm | <300 ppm | <300 ppm |
| (MeOH) | | | | |
| Methyl | <5000 ppm | <500 ppm | <500 ppm | <500 ppm |
| tert-butyl | | | | |
| ether (MTBE) | | | | |
| Tetrahydrofuran | <720 ppm | <72 ppm | 129 ppm | 160 ppm |
| (THF) | | | | |

Microbiological Quantity²

| | | | | |
|---|---|---|---|---|
| TAMC | Max 10³ cfu/g | <100 CFU/g | <100 CFU/g | <100 CFU/g |
| TYMC | Max 10² cfu/g | <100 CFU/g | <100 CFU/g | <100 CFU/g |

¹n-Heptane was only specified for clinical batches.
²Microbiological Quantity was only specified for commercial batches.

### Packing of Dronabinol

Dronabinol was packed in amber Duran PURE bottles for storage and shipment. The bottles were sealed with Duran Pure Premium PTFE Screw caps and sealed under an inert gas; such as argon or nitrogen. Duran bottles were also sealed in a secondary heavy duty mylar bag.

### Storage

The Dronabinol was stored in tight containers that protected from light at ≤25° C. with excursions up to 40° C.

### Example 2: Manufacturing Process for Cannabidiol

### Cannabidiol General Information

International Nonproprietary Names (INN): Cannabidiol

Other Non-proprietary names: A9-Tetrahydrocannabinol, D9-Tetrahydrocannabinol, D9-THC, (−)-trans-D9-tetrahydrocannabinol.

Chemical Names: (−)-Cannabidiol, (−)-trans-Cannabidiol, (−)-trans-2-p-Mentha-1,8-dien-3-yl-5-pentyl-resorcinol, Delta-(2)-trans-Cannabidiol, and 2-[(1R,6R)-3-methyl-6-prop-1-en-2-ylcyclohex-2-en-1-yl]-5-pentylbenzene-1,3-diol.

CAS #: 13956-29-1

Molecular Formula: C₂₁H₃₀O₂

Molecular Weight: 314.47 g/mol

DEA Controlled Substance Schedule: Non-Controlled (determined by USFDA, December 2020).

OEL: estimated to be 5-20 μg/m³

Physical Properties/Appearance: White to slightly beige powder

Solubility: The solubility of cannabidiol in a variety of solvents and oils is summarized in Table 7. The material was found to be insoluble in water and Britton Robinson Buffers (<0.1 mg/mL). The material was found to have a >100 mg/mL solubility (freely soluble) in all other solvents examined with the exception of heptane, n-decane and isooctane.

TABLE 7
Solubility of Various Solvents at 25° C.

| | Solubility | | Solubility |
|---|---|---|---|
| Solvent | (mg/mL) | Solvent | (mg/mL) |
| Water | <0.1 | 1,4-dioxane | >100 |
| n-Decane | 36.8 | 2-butanone | >100 |
| Isooctane | 43.9 | tert-Butyl methyl | >100 |
| | | ether (MTBE) | |
| Heptane | 64.6 | n-hexane | >100 |
| 2-Propanol (IPA) | >100 | N-methyl-pyrrolidone | >100 |
| 1-Propanol | >100 | methylcyclohexane | >100 |
| Anisole | >100 | 2-methyl | >100 |
| | | tetrahydrofuran | |
| | | (2-MeTHF) | |
| Tetrahydrofuran | >100 | 2-methyl | >100 |
| | | tetrahydrofuran | |
| | | saturated with water | |
| Toluene | >100 | Cremophor EL | >100 |
| Cumene | >100 | Tween 80 | >100 |
| Ethyl Formate | >100 | DMSO | >100 |
| Diethyl Ether | >100 | Mineral Oil | 16 |
| 1,2- | >100 | Dodecane Oil | 28 |
| Dimethyloxyethane | | | |
| Acetone | >100 | Sesame Oil | 216 |
| Ethyl Acetate | >100 | Sunflower Seed Oil | >100 |
| (EtOAc) | | | |
| Isobutanol | >100 | BRB pH 2 | 0.00011 |
| Chloroform | >100 | BRB pH4 | 0.00011 |
| Dichloromethane | >100 | BRB pH 6 | 0.00013 |
| Acetonitrile (ACN) | >100 | BRB pH 8 | 0.00012 |
| Isopropyl Acetate | >100 | BRB pH 6 | 0.00013 |
| (IPAc) | | | |
| Cyclohexane | >100 | BRB pH 10 | 0.00013 |
| Methanol (MeOH) | >100 | BRB pH 11 | 0.00041 |
| Ethanol (EtOH) | >100 | Phosphate Buffer pH | 0.00015 |
| | | 6.5 without SLS | |
| p-Xylene | >100 | Phosphate Buffer pH | 0.00012 |
| | | 6.5 with 0.4% SLS | |

BRB = Britton Robinson Buffers;
SLS = Sodium Lauryl Sulfate

### Specific Rotation

The specific optical rotation was measured according to USP <781> on a Perkin-Elmer polarimeter 341. The specific optical rotation was determined for a 10 mg/ml solution of cannabidiol in methanol at 589 nm and 25° C. The specific rotation was measured to be −132.6°.

### Manufacturing of Cannabidiol Drug Substance, Overview of Preparation

The reaction scheme depicting the cannabidiol manufacturing process is shown in the Scheme 2. Olivetol is reacted with bromine in dichloromethane to afford 4,6-dibromoolivetol. 4,6-Dibromo-olivetol is reacted with menthadienol and p-toluenesulfonic acid to afford a solution of dibromocannabidiol (dibromo-CBD) in methanol. Debromination using sodium sulfite and triethylamine affords cannabidiol (CBD), which is recrystallized to yield the cannabidiol drug substance. This step is the last step in the clinical manufacturing process. For the commercial process, Step 4 is an optional step. For the commercial process (only), the CBD is treated with triethylamine. The isolate is recrystallized to afford CBD.

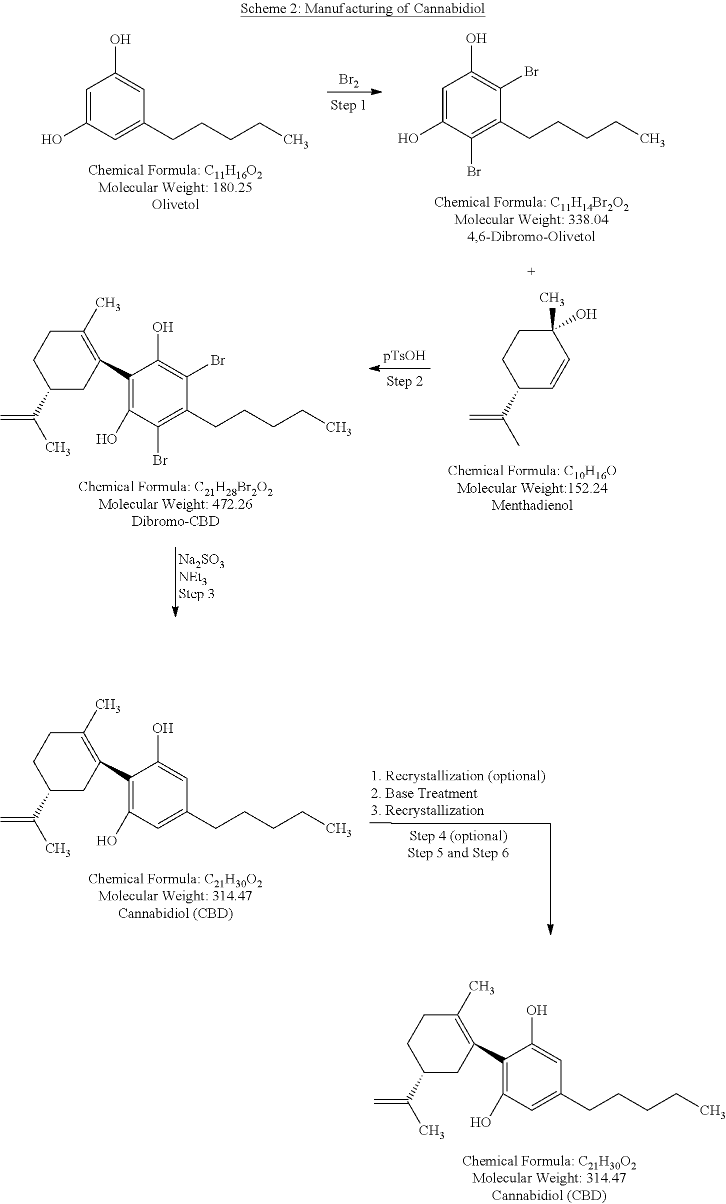

### Specification for Cannabidiol

The proposed specifications for cannabidiol, including test parameters, acceptance criteria and reference to test methods are provided in Table 9. The selected tests and acceptance criteria are intended to ensure the identity, purity, and quality of the drug substance.

TABLE 9
Specification of Cannabidiol

| | | Test |
|---|---|---|
| Test Parameter | Acceptance Criteria | Method |
| 1) Appearance | White to slightly | Visual |
| | beige powder | |
| Identification by IR | Sample Spectra | USP |
| | corresponds | <197> |
| | with reference | |
| Identification by UPL | Retention time | Method in |
| | corresponds with | section |
| | reference | 4.2.1 |
| Water Content (Karl Fisher) | NMT 0.5% | USP <197> |
| Residue on Ignition | NMT 0.2% | USP <281> |
| Chiral HPLC | 99.5% (—)-cannabidiol | 3.2.S.4.2.3 |
| Assay by HPLC | 98.0 to 102.0% w/w | 3.2.S.4.2.3 |
| Assay by UPLC | 95.0%-100.0% w/w | 3.2.S.4.2.1 |
| (Area % Normalization) | | |
| Chromatic Purity (HPLC) | | 3.2.S.4.2.1 |
| Olivetol | NMT 0.15% w/w | |
| 4-Monobromo-Cannabidiol | NMT 0.15% w/w | |
| Delta-9-Tetrahydrocannabinol | NMT 0.10% w/w | |
| Individual unspecified impurity | NMT 0.10% w/w | |
| Total impurities | NMT 1.0% w/w | |
| Residual Solvents (in ppm) | | 3.2.S.4.2.2 |
| 2-Propanol | NMT 5000 ppm | |
| n-Heptane | NMT 5000 ppm | |
| Dichloromethane | NMT 600 ppm | |
| Triethylamine | NMT 5000 ppm | |
| Isooctane | NMT 5000 ppm | |

Compliance with the current USP <232>/<233> elemental impurity requirements has been demonstrated through the elemental impurities analysis of representative plant batches and upon completion of a risk assessment which included an evaluation of raw materials, processing steps, and manufacturing equipment. The manufacturer certifies that this material, if tested, will comply with the established elemental impurity specifications of USP <232>. Methanol was used in clinical manufacturing with a limit of NMT 3000 ppm and not used in the commercial manufacturing process.

### Batch Analysis

Batches of cannabidiol were manufactured at pilot scale or five times pilot scale using the manufacturing process described. General information on these manufacturing batches is presented below in Table 10.

TABLE 10
Clinical (developmental) Batches of Cannabidiol

| | Date of | |
|---|---|---|
| Batch # | Manufacture | Scale |
| 18-NOR-017 | 28 Nov. 2018 | 4.18 kg |
| 18-NOR-018 | 3 Dec. 2018 | 4.46 kg |
| 18-NOR-019 | 4 Dec. 2018 | 3.91 kg |

### Batch Analysis for Clinical Cannabidiol Manufactured

In Table 11 shown below, shows the analytical analysis of the clinical.

| | | 18- | 18- | 18- |
|---|---|---|---|---|
| Batch # | | NOR-017 | NOR-018 | NOR-019 |
| Appearance | White to slightly | Conforms | Conforms | Conforms |
| | beige powder | | | |
| Identification | Sample Spectra | Conforms | Conforms | Conforms |
| by IR | corresponds with | | | |
| | reference | | | |
| Identification | Retention time | Conforms | Conforms | Method in |
| by UPLC | corresponds with | | | section |
| | reference | | | 4.2.1 |
| Water Content | NMT 0.5% | <0.05% | <0.05% | <0.05% |
| (Karl Fisher) | | | | |
| Residue on | NMT 0.2% | <0.05% | <0.05% | <0.05% |
| Ignition | | | | |
| Specific Optical | −140 to −1221 | −128] | −1301 | −132 |
| Rotation | | | | |
| Assay by | 97.0 to | 99.4% | 99.8% | 99.9% |
| HPLC | 102.0% w/w | | | |
| Chromatic Purity (HPLC) | | | | 3.2.S.4.2.1 |
| Olivetol | <0.15% w/w | <0.05% | <0.05% | <0.05% |
| 49- | <0.10% w/w | <0.05% | <0.05% | <0.05% |
| Tetrahydro- | | | | |
| cannabinol | | | | |
| 4-Monobromo- | <0.15% w/w | <0.05% | <0.05% | <0.05% |
| Cannabidiol | | | | |
| Individual | <0.10% w/w | <0.05% | <0.05% | <0.05% |
| unspecified | | | | |
| impurity | | | | |
| Total impurities | <1.0% w/w | <0.05% | <0.05% | <0.05% |
| Residual Solvents (in ppm) | | | | |
| Methanol | 3000 ppm | <300 ppm | <300 ppm | <300 ppm |
| 2-Propanol | 5000 ppm | <500 ppm | <500 ppm | <500 ppm |
| n-Heptane | 5000 ppm | <500 ppm | 500 ppm | 500 ppm |
| Dichloromethane | 1600 ppm | <60 ppm | <60 ppm | <60 ppm |
| Triethylamine | 5000 ppm | <500 ppm | <500 ppm | <500 ppm |
| Isooctane | 5000 ppm | <500 ppm | <500 ppm | <500 ppm |

### Packaging

Cannabidiol drug substance was packaged in two anti-static low-density polyethylene (LDPE) bags (primary packaging material). The inner and outer LDPE bags were twisted and tied with locking wraps. The two LDPE bags were placed inside a high-density (HDPE) drum, pail or suitable secondary container and secured with a tamper evident seal. The primary packaging material fulfills the requirement for packaging pharmaceuticals. All ingredients are compliant to applicable sections of 21 CFR. The polyethylene bags contain an anti-static agent which is approved for use in food grade packaging materials and complies with the requirements of 21 CFR 174-186.

### Storage

The storage instructions for cannabidiol drug substance were determined as follows: “Preserve in tight containers at ≤25° C. with excursions to 40° C.”

### Stability

Manufacturing of all lots of cannabidiol was conducted in a manner consistent with Good Manufacturing Practices and ICH Q7 regulations. Consistent with these principles, and in direct regard to production of cannabidiol, the manufacturer conducted accelerated stability studies utilizing batches of cannabidiol manufactured at full scale. These batches were stored per ICH Q1A (R2) guideline. In addition, the manufacturer has batches of cannabidiol on a long-term stability study. All results obtained have met specification and show no trends towards increased impurity levels or decrease in assay. For these batches, the data and results from these stability studies are in compliance with documentation procedures.

### Example 3: Preparation of Formulation

The formulation may be prepared by mixing CBD, THC, and an excipient at room temperature (˜23° C.). This formulation may be mixed at high speed until a homogeneous oily solution may be obtained.

### Example 4: Proposed Phase 2 Protocol Outline

Active Ingredients used in Study: CBD (cannabidiol)/THC (delta 9 tetrahydrocannabinol).

### Title of the Study

Efficacy of CBD (cannabidiol)/THC (delta 9 tetrahydrocannabinol) formulation versus placebo for the acute treatment of migraine in patients with hemiplegic migraine in a randomized, double-blind, placebo-controlled, multicenter, dose-ranging multiple attack trial with an adaptive dose design.

### Study Center(s)

Multi-site centers may be in U.S. and E.U. Primarily may be a decentralized/digitized trial.

### Objectives

The objection may be to identify a fixed combinations of THC and CBD with optimal efficacy, safety, and tolerability in the treatment of patients with Hemiplegic Migraine to test in a confirmatory phase 3 clinical trial.

### Specific Aims of Phase 2 Study:

### Specific Aim 1:

The first specific aim of the phase 2 study may be to test the hypothesis that THC/CBD may be more effective than placebo in the acute treatment of migraine attacks in patients with hemiplegic migraine.

In this double-blind, randomized, multiple attack trial, subjects may treat 4 separate migraine attacks at least 1 week apart. Co-primary outcomes measured may be headache pain freedom and most bothersome symptom freedom at 2 hours post-dose.

### Specific Aim 2:

The second specific aim may be to test the hypothesis that THC/CBD may be safe and well-tolerated.

We may assess subjective adverse events via smartphone app in real-time as well as vital signs, laboratory tests, electrocardiograms, and examinations prior to treatment #1 and at 24-48 hours post-treatment for the first and fourth doses.

### Specific Aim 3 (Exploratory):

The third specific aim may be to test the hypothesis that THC/CBD may be more effective than placebo in the acute treatment of hemiplegic migraine.

We may assess whether hemiplegia during an attack treated with THC/CBD is alleviated and terminated at a higher rate and more quickly than is hemiplegia during an attack treated with placebo; as this is the first clinical trial to use these outcomes, these will be exploratory outcomes.

### Research Plan for Study

Various sites in this study may be the US and Europe.

### Inclusion Criteria of Participants in Study:

1. Participants may be a male or female ages 18-75.
2. Participants may have a hemiplegic migraine in its familial or sporadic subtypes, as per the International Headache Society (IHS) classification International Classification of Headache Disorders (ICHD-3) criteria (1.2.3).24.
3. Participants may be at least 4 migraines per 18 months.
4. Participants may have the ability to provide informed consent.
5. Participants may agree not to use cannabis outside of the study during participation in the study.
6. Participants may agree not to drive a motor vehicle or operate heavy machinery within 4 hours following last use of cannabis during participation in the study.

### Exclusion Criteria of Participants in Study:

1. Participants may not be pregnant.
2. Participants may not be breastfeeding.
3. Participants may not be a prisoner.
4. Participants may not be urine toxicology positive for THC at screening visit.
5. Participants may not have a known or suspected intolerance to THC, CBD, or other possible excipients of study drug at the discretion of the research team.
6. Participants may not have an ongoing alcohol, opioid or other substance abuse issue.
7. Participants may not have schizophrenia or other severe psychiatric disorder that may be exacerbated by use of THC at discretion of research team.
8. Participants may not have a current or past history of cannabis addiction or abuse.

### Randomization and Blinding:

This study may be a multiple-attack study with a washout period of at least 7 days between treatments. Sample order randomization may be performed by research pharmacist using a random number generator (www.randomizer.org), with patients randomized to verum versus placebo in a 3:1 manner.

A research pharmacist and a statistician may not otherwise be involved with the study and may not review the results weekly, may not decide the dose to be administered to the verum patients for the next block in the adaptive dose design (see below), and may not conduct the randomization and allocation.

The subjects, treating clinicians, all other researchers, and the statisticians performing the overall efficacy analyses may be blinded to the treatment allocations until after statistical analysis is completed.

### Adaptive Dose Design:

An “up-and-down” adaptive dose design algorithm may be used similar to other migraine trials. A block of 8 patients may be randomized to receive either placebo (2 patients) or the starting dose of THC 5 mg/CBD 500 mg (6 patients). If 33% (2 patients) or more of the 4 verum-treated patients in a block may have pain freedom at 2 hours, then the next block of 8 patients to be randomized may be treated with the next lower dose. If less than 33% patients in a block may have pain freedom at 2 hours, then the next block may use the next higher dose. This 33% threshold may be based on the 2-hour pain freedom rates of recent migraine medications for acute care that have received FDA approval.

In this multiple attack study, patients may be randomized 4 times each until each patient may have treated 4 different migraine attacks. The patients may not remain in the block of 8 patients for all 4 treatments as patients may experience migraine attacks at different frequencies. Once a patient treats a migraine attack, they may be placed in a queue again to be re-randomized once a group of 8 patients may be ready to be (re)-randomized.

Once 5 blocks may have been completed at a single dose then the study may proceed into a phase 3 study studying the efficacy of the best performing dose plus one other dose selected by the investigators versus placebo.

### Decentralized and Digitized Trial Design:

S1Tx may intend to apply a decentralized trial design, using an electronic device or smartphone app to collect data, virtual physician assessments and diagnosis, and mobile clinics to collect biometric data, thereby facilitating recruiting and retention and optimizing patient safety in light of the current COVID-19 emergency.

BonTriage, Inc may be a healthcare computing repository and technology company developing digital products to improve chronic medical conditions such as headache, memory problems, sleep issues, etc. It may offer clinical support for headache patients and their physicians to improve outcomes, and to clinical trial sponsors to recruit virtually to lower screen failures, dropouts during the trial, and inadequate data point capture. S1Tx may plan to use BonTriage's two digital products for our trials.

The first engine may be an existing web-based questionnaire accessed by computer or smart phone. Questions from the International Headache Society's Diagnostic Guidelines (ICHD 3) may be asked of patients, then analyzed via an algorithm to produce a document that may read like a neurological consultation, containing a series of clinical impressions and red flags.

For the proposed clinical trial, this may be converted to a shorter, more powerful tool to screen each patient by asking questions in lay language about inclusion and exclusion criteria and to inform and provide clinical decision support to a physician who may independently review the basis for the clinical impressions, consistent with FDA's Draft Guidance on Clinical Decision Support Software, so as to make the diagnosis required for trial participation. (FDA, CDRH, 2019). It may ask factual and psychological questions that enable retention prediction throughout the duration of the trial, limiting screen failures and dropouts, and improving data collection.

### Data Collection:

The second engine may be an existing smart phone app, available in the Apple App store and on Google Play, which may ask similar ICHD 3 questions, may make a clinical impression, and may collect information on ongoing triggers, medications, headaches and other symptoms, and may summarize and trend efficacy of treatment on a daily basis. For a clinical trial, it may be converted into an e-diary which may collect endpoints, medication, and all data necessary for the trial, facilitating patient monitoring while reducing patient and healthcare provider contact and exposure to COVID-19. (FDA, CDRH, 2020)

These digital tools may be HIPPA compliant and may conform to FDA's Guidance, “Use of Electronic Health Record Data in Clinical Investigations” (2018). S1Tx may intend to use these tools to collect patient experience data to inform FDA's benefit-risk assessment.

### Virtual Assessment and Diagnosis:

In order to accommodate potential trial participants with geographical constraints, and to optimize safety in light of the ongoing COVID-19 health emergency, S1Tx may intend to utilize virtual consultation methods, where a physician may perform an assessment (semi structured interview) by video over an electronic device such as a computer or smartphone and may be able to make a diagnosis relevant to the trial criteria.

### Mobile Clinics:

S1Tx may also intend to utilize mobile health services to collect blood draws and urine samples at designated time points during the trial.

### Safety:

As this will be a virtual trial, we may build in good safety monitoring. Our electronic tool may collect some vital sign data; however, we may hire a mobile clinic or trained nurse to visit patients and assess them at home or in the office at various time points. They may take vital signs, EKGs, may perform an examination and may collect laboratory specimen. This may be done during screening and prior to dose 1 of 4, and within 48 hours after dose 1 and after dose 4.

### Tolerability:

Patients may be asked multiple times starting after they sign the informed consent if they may have developed any new symptoms or illnesses or required medical attention or hospitalization. This may be done via the app. All adverse effects, whether or not judged by the physician to be due to the investigational medication, may be recorded.

### Procedure:

Subjects may be asked to initiate treatment as early as possible in the course of a migraine attack once the pain intensity may have reached a 2 (moderate) or 3 (severe) intensity on a 0-3 scale providing:
1. It is within the first 2 hours of this migraine attack.
2. No other pharmacologic co-intervention (prescription, over-the-counter, cannabis or other) may have been used for this migraine attack.
3. The migraine pain may not resolve on its own.

Subjects may have up to 12 months to treat 4 separate migraine attacks. Subjects may be instructed to wait for a washout period of at least 7 days (including at least 48 hours headache-free) between treated migraine attacks.

### Study Drug:

We may use an adaptive dose design as described above. Our anticipated starting dose may be THC 5 mg/CBD 500 mg, and our anticipated doses to study may range among the following 7 doses:
1) THC 1.25 mg/CBD 500 mg
2) THC 2.5 mg/CBD 500 mg
3) THC 5 mg/CBD 500 mg
4) THC 7.5 mg/CBD 500 mg
5) THC 10 mg/CBD 500 mg
6) THC 15 mg/CBD 500 mg
7) THC 20 mg/CBD 500 mg
8) Placebo (devoid of cannabinoid content;)

Each subject may be provided a lock box in which to store the treatments assigned to them.

### Assessments:

Subjects may complete assessments using a mobile phone application digital assessment tool which may prompt subjects to enter data at 8 time points (15 minutes, 30 minutes, 45 minutes, 1 hour, 2 hours, 4 hours, 24 hours, and 48 hours).

### Endpoints:

Our pre-specified co-primary endpoints may be headache pain freedom and most bothersome symptom at 2 hours. Migraine endpoints may be selected based on the FDA's publication “Migraine: Developing Drugs for Acute Treatment-Guidance for Industry. The FDA recommends, “Migraine-associated headache pain and associated symptoms may be measured by asking patients to self-report the current status of their headache pain and associated symptoms. A four-point Likert scale may be used for headache pain (i.e., 0=none, 1=mild, 2=moderate, 3-severe), and a binary scale (present or absent) may be used for associated migraine symptoms.” As this may be the first clinical trial conducted for the treatment of hemiplegic migraine, our hemiplegic migraine endpoints may be exploratory and adapted from an open-label study of intranasal ketamine for hemiplegic migraine.

Cannabis-related safety, tolerability, and other endpoints may be based on prior RCTs of cannabis for the treatment of pain. Subjects may be asked to report euphoria (Y/N), sleepiness (Y/N), subjective impairment (Y/N), and highness (0-10 Visual Analog Score). “Euphoria” is a pleasant subjective feeling associated with drug liking, while the term “highness” may be used to describe subjective impairment that may or may not be pleasant and may not be associated with drug liking. Subjective psychoactive side effects may be added as covariates to the model to assess whether pain response remains significant above and beyond the subjective psychoactive side effects.

### Co-Primary Efficacy Endpoints:

1. Headache pain freedom at 2 hours (answer 0=no pain, from options of 0=no pain, 1=mild pain, 2=moderate pain, and 3=severe pain).

Most bothersome symptom freedom at 2 hours (answer Y/N to question of presence of symptom from photophobia, phonophobia, or nausea identified as most bothersome symptom at time 0).

### Secondary Efficacy Endpoints

2. Headache pain freedom at 15 minutes, 30 minutes, 45 minutes, 1 hour, 4 hours, 24 hours, and 48 hours (Y/N: reduction from 2 or 3 pre-treatment to 0 or 1 post-treatment {not 1 for pain freedom only for pain relief} on 0-3 scale, with 0=no pain, 1=mild pain, 2=moderate pain, and 3=severe pain).
3. Headache pain relief at 15 minutes, 30 minutes, 45 minutes, 1 hour, 2 hours, 4 hours, 24 hours, and 48 hours (Y/N) 3 or 2 to 1 or 0.
4. Most bothersome symptom freedom at 15 minutes, 30 minutes, 45 minutes, 1 hour, 2 hours, 4 hours, 24 hours, and 48 hours (Y/N).
5. Freedom from photophobia at 15 minutes, 30 minutes, 45 minutes, 1 hour, 2 hours, 4 hours, 24 hours, and 48 hours (Y/N).
6. Freedom from phonophobia at 15 minutes, 30 minutes, 45 minutes, 1 hour, 2 hours, 4 hours, 24 hours, and 48 hours (Y/N).
7. Freedom from nausea at 15 minutes, 30 minutes, 45 minutes, 1 hour, 2 hours, 4 hours, 24 hours, and 48 hours (Y/N).
8. Use of any rescue medication (Y/N) at 4 hours, 24 hours, and 48 hours.
9. Sustained pain freedom (no headache pain at 2 hours after dose, with no use of rescue medication and no relapse of headache pain) at 24 hours and 48 hours (Y/N).

### Exploratory Hemiplegic Migraine Endpoints

10. Freedom from hemiplegia at 15 minutes, 30 minutes, 45 minutes, 1 hour, 2 hours, 4 hours, 24 hours, and 48 hours (Y/N).
11. Arm hemiplegia severity may be at 15 minutes, 30 minutes, 45 minutes, 1 hour, 2 hours, 4 hours, 24 hours, and 48 hours (on a 0-3 scale, with 3=not able to move arm, 2=able to move arm but not hold item, 1=able to hold item but not lift arm overhead, and 0=able to hold item and lift arm overhead).
12. Leg hemiplegia severity at 15 minutes, 30 minutes, 45 minutes, 1 hour, 2 hours, 4 hours, 24 hours, and 48 hours (on a 0-3 scale, with 0=not able to move leg, 1=able to move leg but not stand, 2-able to stand but not able to walk, and 3=able to walk).
13. Any hemiplegia severity at 15 minutes, 30 minutes, 45 minutes, 1 hour, 2 hours, 4 hours, 24 hours, and 48 hours (lowest number responded to either Arm hemiplegia (#13) or Leg hemiplegia (#14) 38.

### Safety, Tolerability, and Other Endpoints

14. Euphoria (Y/N).
15. Sleepiness (Y/N).
16. Impaired (Y/N).
17. Highness (0-10 VAS).

### Rescue Treatments:

Rescue treatments may be permitted at 2 hours after administration of the study drug after the patient completes the 2-hour co-primary outcome reporting. Patients who do not have pain freedom at 2 hours may be allowed to take a second dose of the same cannabis treatment or rescue with their home medications. This medication use may be recorded, and responses will be recorded at 4 hours.

### Risk Management:

As part of the training session, subjects may be provided with a handout about potential side effects and self-management in the case of excessive psychoactive side effects (including deep breathing and watching television while waiting for the effects to wear off). A member of the research team may be available 24 hours a day, 7 days a week, to receive pages from study subjects should immediate concerns arise.

### Safety:

Since this may be a virtual trial, we may build in good safety monitoring. Our electronic tool may collect some vital sign data; however, we may hire a mobile clinic or trained nurse to visit patients and may assess them at home or in the office at various time points. They may take vital signs, EKGs, do an examination and collect laboratory specimen. This may be done during screening and prior to dose 1 of 4, within 48 hours after dose 1 and dose 4.

### Tolerability:

Patients may be asked multiple times via the app, starting after they sign the informed consent, if they may have developed any new symptoms or illnesses or required medical attention or hospitalization. This may be done via the app only. All adverse effects, whether or not judged by the physician to be due to the investigational medication, may be recorded.

### Sample Size:

The sample size may be determined based on detecting a 20% difference between verum and placebo in our co-primary outcomes at 2 hours. To detect a 20% difference between verum and placebo may require a sample size of n=64 to detect with 80% power and a two-sided type I error rate alpha=0.05. We may plan to enroll n=80 to account for dropouts.

### Statistical Plan:

To compare the treatment and placebo groups, the model may be a co-primary as well as each of the secondary outcomes using the semi-parametric generalized linear models, the weighted generalized estimating equations (WGEE), with an appropriate link function depending on the type of response (e.g., logit for the co-primary binary outcome), in which treatment groups (time-varying binary indicators) may be the predictors. Unlike traditional applications of WGEE, each cluster may consist of data from four treatment groups and repeated assessments within each treatment group from each individual, because of the multiple-attack design of the study. If there may be significant differences among demographic and migraine-related variables at baseline, they may be controlled for in the model as covariates. WGEE may require no mathematical distribution such as normality. Further, as dropout may not be random, i.e., may not follow the missing completely at random (MCAR) assumption and may affect inference for treatment differences because of the multiple-attack design, use of WGEE not only may provide valid inference for a broader class of data distributions but also under the more general missing at random mechanism (MAR). Treatment difference may be tested using appropriate linear contrasts. Clustered bootstrap inference may also be applied and reported, if discrepancy occurs between the bootstrap and asymptotic inference, as the usual asymptotic inference may be limited by the sample size. By redefining clusters to include all assessments (at 15 minutes, 30 minutes, 45 minutes, 1 hour, 2 hours, 4 hours, 24 hours, and 48 hours), the above approach may also be applied for inference about the secondary outcome.

### Example 5: Effects of CBD:THC on CSD Susceptibility and CSD-Related Behavior in WT and FHM1 Mutant Mice

The overall objective of this study was to determine the efficacy of an acute dosage of CBD:THC at a ratio of 30:1 (30 mg/kg CBD: 1 mg/kg THC) or a ratio of 100:1 (100 mg/kg CBD: 1 mg/kg THC) on suppressing CSD and migraine-related features in wildtype (WT) and FHM1 KI mice to assess the therapeutic effect. All experiments were performed in awake freely moving mice.

For Aims 1 & 2, CSDs were evoked in WT and FHM1 mice to investigate whether CBD:THC suppresses CSD features and CSD-related ‘mouse grimace scale’ as a migraine-relevant behavioral feature. Optogenetics was used to induce CSD as this method allows for minimally invasive induction of CSD thus without confounding effects of anesthesia. For Aim 3 the effects of CBD:THC on the hindpaw withdrawal threshold were investigated in WT and FHM1 mice. In all aims, mice of both sexes were studied.

### Aim 1: Effects of CBD:THC on CSD Characteristics in WT and FHM1 Mice

The objective was to determine whether an acute dose of i.p. injected CBD:THC normalizes CSD properties (i.e. threshold, propagation rate and basic features amplitude and width) with possible stronger effects in FHM1 compared to WT mice. CSD threshold experiments were performed in Thy1-ChR2 (Thy1-driven expression of channelrhodopsin 2)/FHM1 and Thy1-ChR2/WT mice.

### Aim 2: Effects of CBD:THC on CSD-Related Head Pain Behavior in WT and FHM1 Mice

The objective was to determine whether an acute dose of i.p. injected CBD:THC normalizes CSD-related head pain behavior, with possible stronger effects in FHM1 mice compared to WT mice. Mice of both sexes were studied. In the context of the duration of treatment effects, head pain behavior was assessed both acutely 30 minutes after CSD, as a measure of headache initiation, and at 24 and at 48 hours after CSD, as a measure of headache duration.

### Test Animals and Systems

### Aims 1 & 2: Thy1-ChR2/WT and Thy1-ChR2/FHM1 Mice

Thy1-ChR2/YFP/WT mice (both genders-age range at day of surgery 6-13 months).

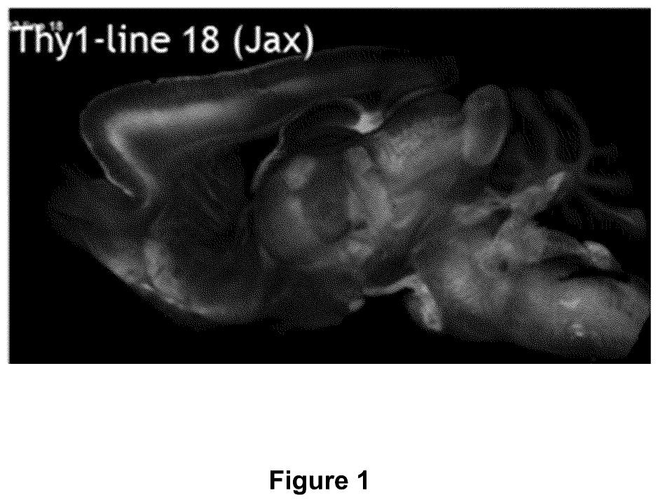

Strain 7612—B6.Cg-Tg (Thy1-COP4/EYFP) 18Gfng/J; Thy1/ChR2-YFP from the Jackson Laboratory, Bar Harbor, ME, USA (see FIG. 1).

### Test Animal Housing and Care

Mice were kept under standard housing conditions (temperature of 22+1.5° C., 12-h light/12-h dark cycle) and had free access to water and food. After recovery from surgery, mice were individually housed in a Faraday cage with video-EEG setup, in which they were connected to a 7-channel counterbalanced commutator for the continuous recording of video and DC- and AC-EEG. During recordings, animals could move freely.

Experiments were approved by the local and national ethical committees according to ARRIVE guidelines and recommendations of the European Communities Council Directive (2010/63/EU). All efforts were made to minimize discomfort of the experimental animals.

### Materials

Surgery equipment: Heating pad (CMA/150 Temperature Controller Carnegie Medicine); Stereotactic-device (David Kopf instruments model 900 series); Surgical Lamp (Schott KL 1500 compact); Dry sterilizer (Germinator 500); Drill (Drill bit size 0.5, FST #19008-05); Zeiss surgery microscope; Surgery tools; Isoflurane gas anesthetic setup.

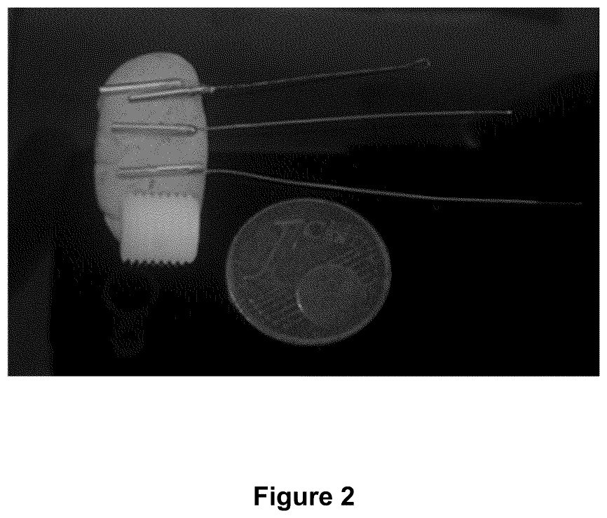

Electrodes (see FIG. 2): Aim 1: Platinum90/Iridium10 wire, quadruple PTFE insulated (Ø 0.075 mm; PT67811, Advent Research Materials); Aim 2: Silver wire, quadruple PTFE insulated (Ø 0.075 mm; AG5493, Advent Research Materials); Electrode socket contacts (E363/0, Plastics One)

Optic fibers (Aims 1 & 2): Fiber Optic Cannula, Ø2.5 mm SS Ferrule, Ø 400 μm Core, 0.39 NA, L=2 mm (CFM14L02, Thorlabs).

Dental Cement: Sun Medical superbond C&B (with L-polymer; 075794, Hofmeester, the Netherlands).

DC-EEG-setups used for Aims 1 & 2: Custom made Faraday cage for multi-site DC-EEG measurements; 7-channel counterbalanced swivel; Passive Infrared (PIR) Motion sensor; Drinking sensor; Front-view video-camera; Custom-made pre-amplifiers; Digitizer (Power 1401 Mk3, Cambridge Electronic Design, CED); PC with Spike2 software (CED).

LED system used for optogenetic CSD induction (Aims 1 & 2): 460-nm LED light source (UHP-T-LED-460; Prizmatix); Calibration equipment for optic fibers (PM100A with S121C photodiode, Thorlabs).

Behavioral system for MGS assessment (Aim 2): MGS acrylic cylinder (10 cm width, 15 cm height); 4K-sony HD-camera (HDRCX625B, Sony).

Compounds: Stock CBD: 100 mg/ml (0.2 ml/vial; in sunflower oil, from Eurofins) stored at room temperature; Stock THC: 1 mg/mL (0.5 ml/vial; in sunflower oil, from Eurofins) stored at room temperature.

CBD:THC working solutions were prepared one or at maximum two days prior to the experiment. All working solutions were prepared in 2 mL glass vials (obtained from Eurofins CDMO; clear glass VCDIN2RDLS1, Schott, France) with rubber stopper coated with flurotec bromobuyl (from Eurofins CDMP; INJ13TB3WRSDL1, 13 mm Westar RS, France). For preparations, standard plastic pipette tips were used, taking into account slow pipetting rates given high viscosity of solutions. Working solutions of 100:1 or 30:1 CBD:THC were prepared by dilution with sunflower oil (Fagron), from stocks of 100 mg/mL CBD (Eurofins) and 1 mg/mL THC (Eurofins), respectively. Order of pipetting followed the concept to add the highest concentration compound to the lowest, to ensure proper mixing; this implies that first THC stock was added to the vehicle, followed by CBD. Working solutions were gently mixed overnight at room temperature on a shaking platform at 100 rpm. For all i.p. injections in mice, plastic 1 ml syringes with a 25G needle were used.

### Methods

### Aim 1-CBD:THC Effects on CSD Threshold and Other CSD Susceptibility Features

Summary of approach-Mice of both genders were studied. CSD threshold experiments were performed in Thy1-ChR2 (Thy1-driven expression of channelrhodopsin 2)/FHM1 and Thy1-ChR2/WT mice. In the experiments, optic fibers for optogenetic CSD induction were placed on the skull. In first experiments, a dural optic fiber was also placed to assess whether this may yield more stable threshold values which turned out not to be the case. Recording electrodes were placed intracortically to accurately assess CSD propagation rate, amplitude, and width. For each mouse, first, threshold measurements were performed on multiple days to establish a stable baseline, before testing effects of CBD:THC. To reduce the number of mice and maximize the outcome of the experiments, multiple doses and vehicle were tested in each mouse for a period of 3-4 weeks.

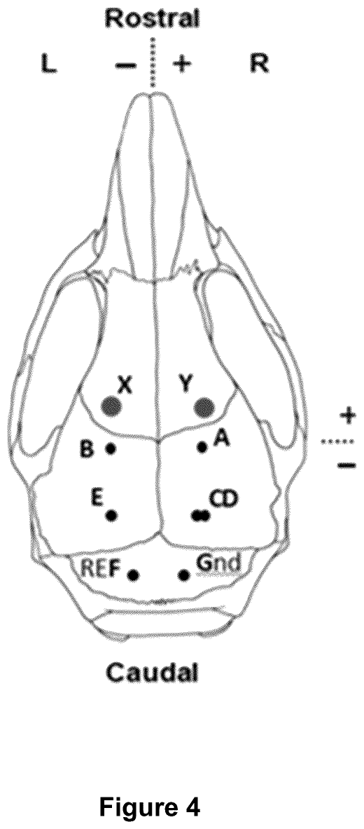

Surgery-Electrodes (single or paired 75 μm Pt/Ir) were implanted under isoflurane anaesthesia (4-5% induction, 1.7-2% maintenance) in oxygen-enriched air, with body temperature maintained around 37° C. by a rectal probe and homeothermic blanket control unit, at the following coordinates (mm to bregma): 3.5 posterior/2.0 lateral/0.6 depth (right V1); 1.0 anterior/2.0 lateral/0.8 depth (right M1); 3.5 posterior/2.0 lateral/0.6 depth (left V1); 2 electrodes (with 1-mm uninsulated tips) in the cerebellum served as reference and ground. An optic fiber cannula (400 μm, Thorlabs) for photo-stimulation was placed on the skull bone over right and left M1 (0.8 mm anterior and 2.0 mm lateral to bregma; see X and Y in FIG. 4). Electrodes (connected to a seven-channel pedestal, see FIG. 4) and optic fibers were attached to the skull using dental cement (Sun Medical superbond C&B, with L-polymer; #075794, Hofmeester, the Netherlands). Carprofen (5 mg/kg, s.c.) was administered for post-operative pain relief. Note: first series of Aim 1 experiments were performed using Diadent cement, which proved unstable, and was thus not used for the drug tests with serial design (see also Results).

Electrophysiological recordings-One week after surgery, mice were transferred to a shielded electrophysiological set-up for freely behaving mice, as previously described (Houben et al., 2017). The system consisted of a Faraday cage, in which the mouse was connected to custom-built recording hardware through a counterbalanced, low-torque electrical commutator. Electrophysiological signals were 3× pre-amplified and fed into separate amplifiers for direct current (DC)-potential (500 Hz low-pass; 10× gain) and alternating current (AC)-potential (0.05-500 Hz; 800× gain), respectively. Signals were digitized (Power 1401 and Spike2 software, CED, Cambridge, UK) at a sampling rate of 1,000 Hz (DC-potential) or 5,000 Hz (AC-potential). Differential signals from paired cortical electrodes were used to detect multi-unit activity (MUA; 500-5,000 Hz; 36,000× amplification; 25,000 Hz sampling rate).

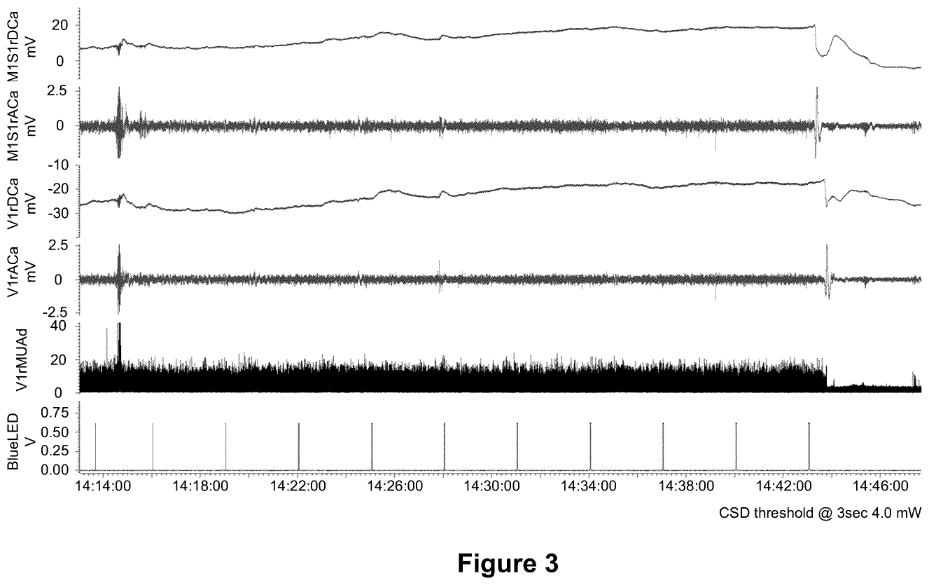

CSD threshold assessment-Optogenetic stimulation experiments were started after 1 day of baseline ECoG and MUA recording, see example scheme below. CSD threshold assessment was repeated on separate days in random order, up to one month after surgery. A 460-nm LED light source (UHP-T-LED-460; Prizmatix) was connected to the optic fiber for blue light stimulation in the electrophysiology set-up. Delivered power at optic cannula tip was calibrated to 4 mW before the experimental series (PM100A with S121C photodiode; Thorlabs). For assessing CSD threshold, single 4 mW blue light pulses were applied to the right M1 (in seconds: 0.3, 0.6, 0.9, 1.2, 1.5, 1.8, 2.1, 2.4, 2.7, 3, 4, 5, 6, 7, 8, 9, 10, 12, 15, 20, 30) until CSD was observed. If 4 mW pulses were unable to induce CSD, 30 seconds pulses with increasing intensity (up to 12 mW) were used. In case photo-stimulation by the right M1 optic fiber was not able to evoke CSD, the left M1 optic fiber was used. See example recording during threshold induction in FIG. 3.

Pharmacology-One to two days before the experimental day, fresh working solution was prepared for dosages of 100:1 CBD:THC or 30:1 CBD:THC. The working solution was gently mixed on a rocking table overnight at room temperature. Injection volume was based on the body weight of an animal; per gram body weight, 6 μl CBD:THC working solution was injected. Compounds were tested in a longitudinal design, to include recovery days and vehicle tests alternating the drug administrations. Repetition of drug tests were included in the first series of experiments as reproducibility control. Drugs or vehicle (sunflower oil) were administered i.p., at 30 minute or 1 hour prior to the start of the first photo-stimulation. Before assessing effects of CBD:THC on CSD threshold and other features, 3 baseline CSD threshold tests were performed to ensure stable values throughout the longitudinal design. Following drug administration, at least 1 resting day was taken into account for washout of the administered compounds. See example schedule in Table 12 below.

TABLE 12
Week 1

| Monday | Tuesday | Wednesday | Thursday | Friday |
|---|---|---|---|---|
| | | Basal TH | Basal TH | Basal TH |

Week 2

| Monday | Tuesday | Wednesday | Thursday | Friday |
|---|---|---|---|---|
| Vehicle/ | CBD:THC | Rest day | CBD:THC | Rest day |
| 1 hr | 100:1 | | 100:1 i.p./ | |
| wait/TH | i.p./1 hr | | 30 min | |
| | wait/TH | | wait/TH | |

Week 3

| Monday | Tuesday | Wednesday | Thursday | Friday |
|---|---|---|---|---|
| Basal TH | Basal TH | CBD:THC | Rest day | CBD:THC |
| | | 30:1 | | 30:1 |
| | | i.p./1 hr | | i.p./30 min |
| | | wait/TH | | wait/TH |

Week 4

| Monday | Tuesday | Wednesday | Thursday | Friday |
|---|---|---|---|---|
| Basal TH | Basal TH | | | |

### Aim 2-CBD:THC Effects on CSD-Induced MGS

Summary of approach—To minimize confounding effects by invasive surgery on head pain behavior, for this aim both the optic fiber for optogenetic CSD induction and the recording electrodes were placed on the skull. Three CSD events were induced by suprathreshold intensity light stimulation. Pain-relevant behavior was quantified using a home-modified version of the mouse grimace scale (MGS, a behavioral measure of spontaneous mimics related to head-pain; (Langford et al., 2010)). We investigated, in each mouse, the effect of an acute dose of CBD:THC by MGS measured after 30 minutes, 24 hours, and 48 hours to also take into account possible effects on headache duration.

Surgery—Mice underwent minimally-invasive surgery using isoflurane anaesthesia (4-5% induction, 1.7-2% maintenance) in oxygen-enriched air, with body temperature maintained around 37° C. by a rectal probe and homeothermic blanket control unit. Small and superficial indentations were drilled on the skull bone without penetrating the skull over the somatosensory (S1) and visual cortex (V1), in which silver (Ag, with ˜100 μm diameter ball tip) electrodes were fixed to the skull using graphite wire glue (Anders Products, Melrose MA, USA) at the following coordinates (mm to bregma): 1.0 anterior/1.0 lateral (right M1); −1.0 posterior/1.0 lateral (right S1); −3.0 posterior/1.0 lateral/(right V1). Two Ag ball-tip electrodes on the skull at the cerebellum served as reference and ground. A fiber optic cannula (400 μm; CFM14L02, Thorlabs, Newton NJ, USA) for photo-stimulation was placed on the skull bone over right V1 cortex (3.5 mm posterior and 2.0 mm lateral to bregma). Electrodes were connected to a pedestal and attached to the skull with dental cement as described for Aim 1 experiments.

Recording and induction of CSD—After a seven-day recovery period, animals were placed in the EEG recording system as in Aim 1 experiments. Electrophysiological signals were pre-amplified 3× and fed into separate amplifiers for direct-current (DC) potential (DC-500 Hz low-pass filter, 10× gain, relative to reference) and LFP (0.05-500 Hz band-pass filter, 1200× gain, relative to reference). Signals were digitized (Power 1401 with Spike2 software; CED, Cambridge, UK) at sampling rates of 1000 Hz for DC-potential and 5000 Hz for LFP signals. After a 10 minute habituation to the set-up and verification of stable DC-potential signals, three consecutive CSDs, separated by 5 minutes, were induced in the V1 location by optogenetic stimulation using a suprathreshold pulse of 460 nm blue light of 4-mW intensity at 30 seconds duration. In cases were no CSD could be induced by a given photo-stimulation, an additional stimulation at higher intensity was given to ensure 3 successful CSD inductions within a 20-minute period. CSD events were identified by their characteristic DC-deflections and spread between the 2 recording electrodes.

Video-recording of mouse facial mimics—Video recordings were used to obtain MGS scores before and after CSD. To this end, the mouse was placed within a glass jar (10 cm width, 15 cm height) that allowed free movements (i.e. without restriction of full body movements such as rearing and grooming). The jar was placed in front of the CSD induction/recording cage with a Full HD camera with 1920×1080 resolution (HDRCX625B, SONY) placed at the front. Videos were recorded at 25 fps. MGS was analyzed post hoc from video for the 10 minutes before the first photo-stimulation (baseline) and for the 30-40 minute window following the first CSD (i.e. 20-30 minute window following the last CSD). To consider possible longer-term effects of chronic drug treatment, MGS was also assessed at 24 h and 48 h in the absence of CBD:THC. To rule out that the tower (containing the ball tip electrodes and optogenetic fiber cannulas) on the head of the mouse may confound the assessment of pain-related behavior, mice with and without such tower had been tested in the glass jar during pilot experiments, which showed that all MGS features could be reliably assessed in mice with a head tower. The head pain monitoring set up was cleaned with ethanol in between sessions with different animals, but not when it concerned different sessions of the same animal.

Pharmacology—CBD:THC working solutions were prepared as indicated for Aim 1 experiments. Compounds were tested in a longitudinal design, to include recovery days and vehicle tests alternating the drug administrations. Repetition of drug tests were included in the first series of experiments as reproducibility control. Drugs or vehicle (sunflower oil) were administered i.p., at 30 minutes prior to the start of the first CSD induction. Following drug administration, at least 1 resting day was taken into account for washout of the administered compounds. See example schedule below in Table 13.

TABLE 13
Week 1

| Monday | Tuesday | Wednesday | Thursday | Friday |
|---|---|---|---|---|
| | Baseline/MGS | MGS 24 h | MGS 48 h | |
| | following | after | after | |
| | opto- | opto-CSD | opto-CSD | |
| | CSD | | | |

Week 2

| Monday | Tuesday | Wednesday | Thursday | Friday |
|---|---|---|---|---|
| | Vehicle/MGS | MGS 24 h | MGS 48 h | |
| | following opto- | after | after | |
| | CSD | opto-CSD | opto-CSD | |

Week 3

| | | | | |
|---|---|---|---|---|
| Monday | Tuesday | Wednesday | Thursday | Friday |
| | CBD:THC | MGS 24 h | MGS 48 h | |
| | 100:1 I.p. | after | after | |
| | MGS following | opto-CSD | opto-CSD | |
| | opto-CSD | | | |

Week 4

| Monday | Tuesday | Wednesday | Thursday | Friday |
|---|---|---|---|---|
| | CBD:THC 30:1 | MGS 24 h | MGS 48 h | |
| | I.p./MGS | after | after | |
| | following opto- | opto-CSD | opto-CSD | |
| | CSD | | | |

### Data Analysis

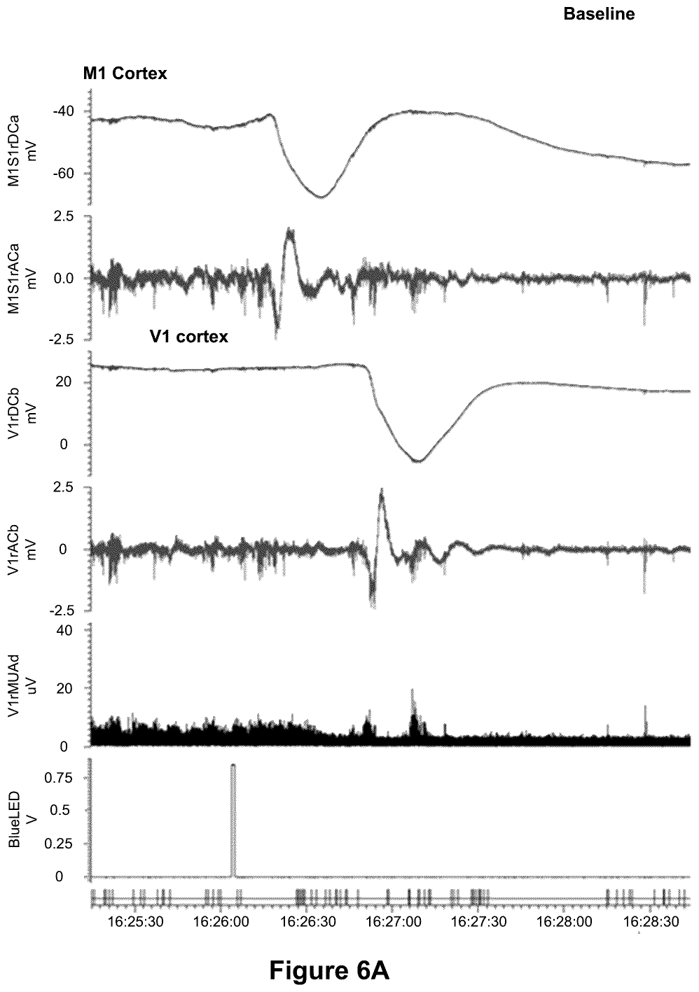

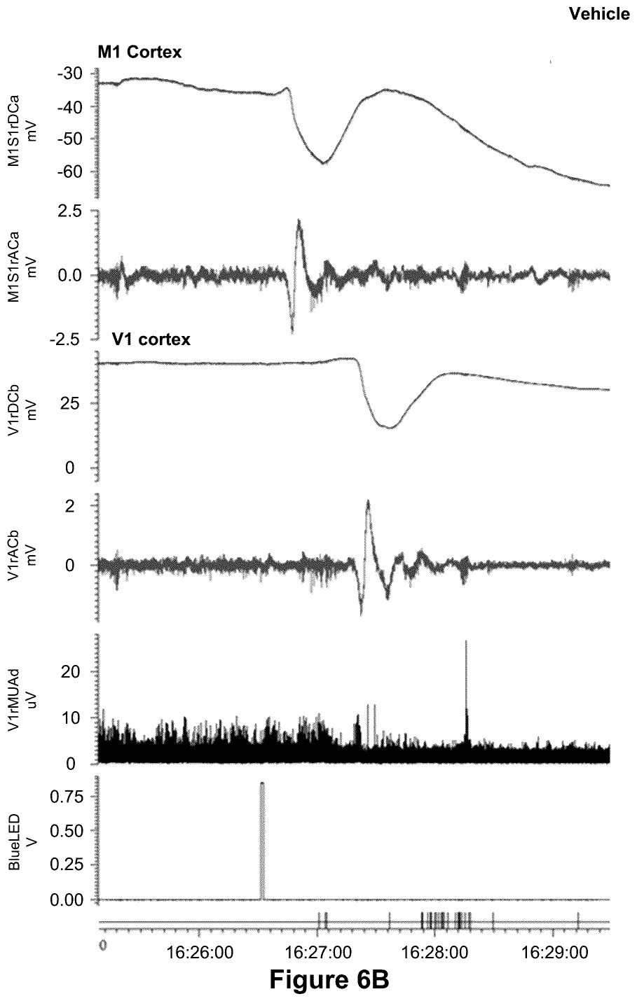

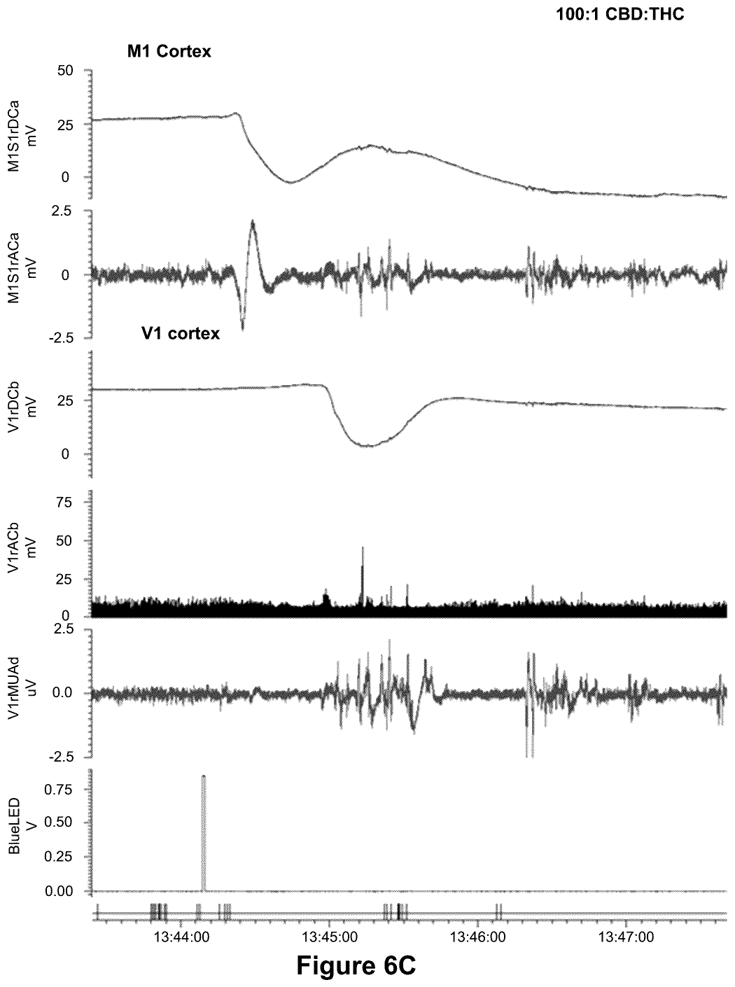

CSD occurrence and features (Aim 1)—DC-EEG data were analysed off-line using Spike2 software. CSD was defined as a transient negative DC-shift with amplitude >5 mV measured with a delay at two recording locations and accompanied by a depression of local MUA and/or associated with a decrease in AC amplitude (see examples in FIG. 3 and FIGS. 6A-6C).

MGS analysis (Aim 2)—The MGS method independently scores five facial action units (FAUs) (Langford et al., 2010) on a 3-point scale for their presence and intensity as follows: a value of 0 (not present), 1 (moderately visible) or 2 (severe). The five FAUs concern: orbital tightening, ear position, nose bulge, cheek bulge and whisker position. An averaged MGS score was obtained for each mouse for each time point by averaging the 5 FAUs. MGS was assessed post hoc, by scoring a 1-minute video frame that was judged to represent best the MGS behavior of the 10-minute video To this end, each video was first observed as a whole, after which a 1-minute time frame was chosen that was judged to best represent the MGS observed throughout the whole video. The post hoc observer was blinded to the genotype and experimental groups as to prevent a possible bias when attempting to score facial expressions.

Statistical analysis-Data visualization and statistical testing were performed using Graphpad Prism (GraphPad Software). Data were presented as mean±standard error of the mean (SEM) and analysed using a Wilcoxon signed-rank test.

### Results

### Aim 1: Effects of CBD:THC on CSD Characteristics in WT and FHM1 Mice

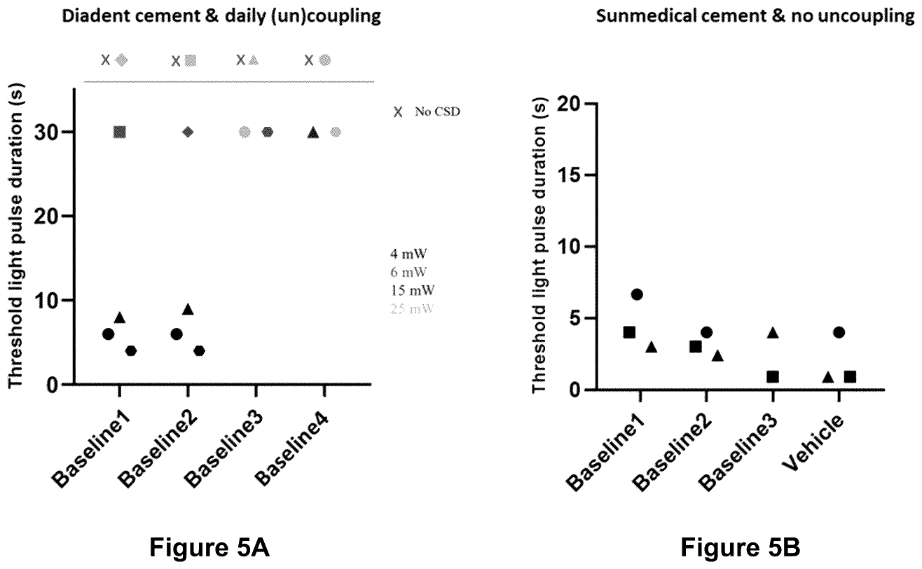

Repeated CSD threshold experiments for serial drug tests in freely behaving mice require extremely stable EEG head-mounts—The first series of CSD threshold experiments were performed with Diadent chemical cement, and mice were coupled/uncoupled to the EEG setup at each experimental day, to minimize the number of EEG setups required for the experiments. Using this approach, repeated assessment of baseline CSD thresholds revealed instable thresholds at 4 mW photo-stimulation, with mice requiring higher intensities or not developing a CSD event at the highest intensity used of 25 mW (FIG. 5A). In addition, several mice lost the Diadent-based head-mount during the course of the experimental series, thereby not making it through or beyond the baseline period. Switching to a different chemical cement type, Sun Medical, yielded well-fixed head-mounts during the course of the 3-4 week experiments, and stable baseline CSD threshold values (FIG. 5B). All experiments with CBD:THC were therefore performed using Sun Medical cement for the EEG head-mounts, and keeping mice connected to the setup throughout the experimental series. Mice were only included for CBD:THC tests if baseline CSD threshold values tested over the course of 3 days proved stable.

Acute pretreatment by CBD:THC does not affect CSD susceptibility features for optogenetically-induced CSDs-Using Sun Medical cement, and keeping each mouse connected to the EEG setup throughout the entire 3-4 week experimental period, effects of acute pretreatment with a dosage of CBD:THC (administered i.p. 1 hour or 30 minutes prior to the start of photo-stimulation for optogenetic CSD induction)—were tested on CSD features threshold, propagation rate, halfwidth and amplitude. FIGS. 6A-6C show example CSD recordings during baseline, vehicle and CBD:THC pretreatment, illustrating successful induction of CSD events in the presence of CBD:THC.

A total of N=11 WT, and N=12 FHM1 mice enrolled in the Aim 1 experiments, of which successful Aim 1 datasets could be obtained for N=6 WT and N=7 FHM1 animals, which concerns data from a total of more than 150 daily CSD threshold tests. Considering the various CSD susceptibility features, not all animals provided information on all CSD features threshold, propagation, half-width and amplitude. Dropouts were due to technical issues related to i) instable head-mount, ii) instable electrode/optic fiber connections, iii) unconventional CSD events hampering CSD feature analysis.

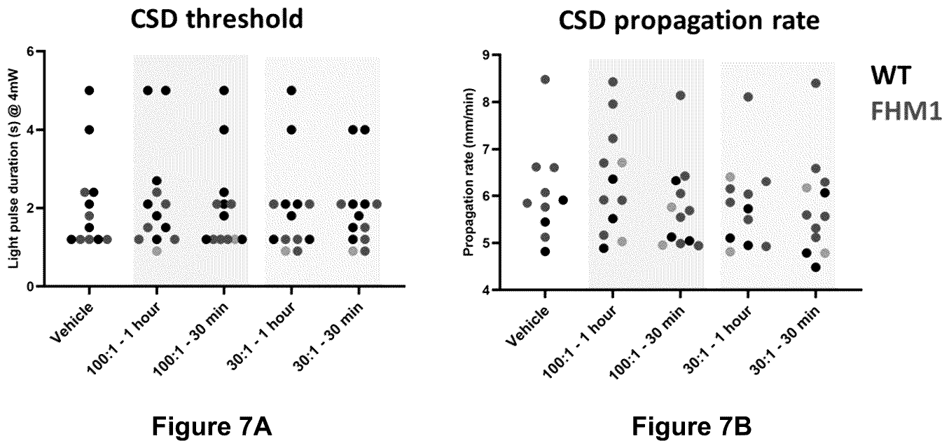

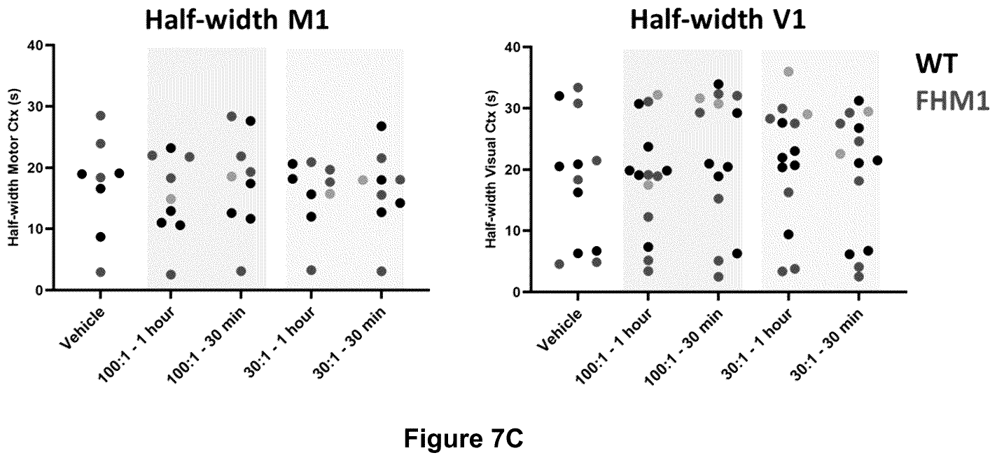

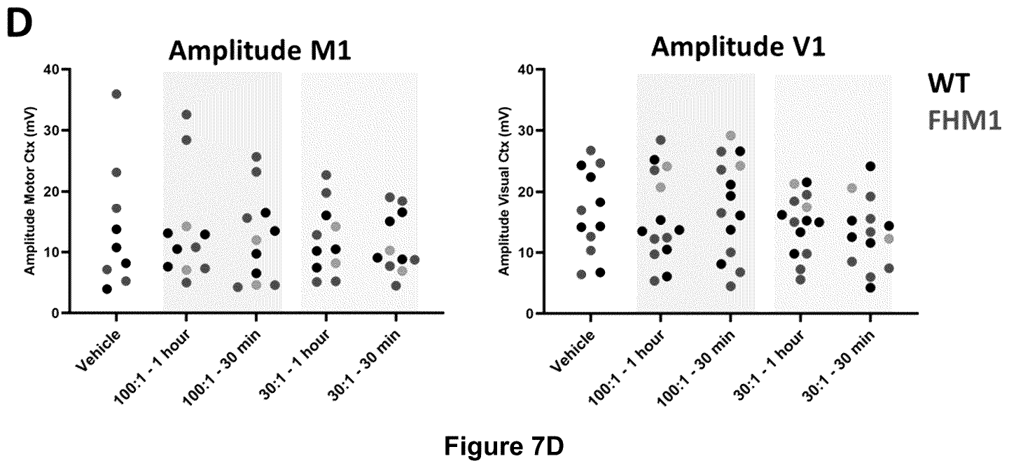

FIG. 7A shows the opto-CSD threshold results from WT and FHM1 mice, with none of the dosages tested (100:1 or 30:1 CBD:THC) causing a change in CSD threshold compared to the vehicle control condition. FIG. 7B shows that also CSD propagation rate was not affected by the CBD:THC pre-administration. The reported increase in CSD propagation rate as reported for FHM1 compared to WT mice in earlier studies for KCl-induced CSD in anesthetized animals (Eikermann-Haerter et al., 2009) was evident throughout the experimental series with typically higher rates for FHM1 mice compared to WT values. This underscores the relevance of including tests of CSD propagation rate in FHM1 mice compared to WT mice as a readout for CSD susceptibility, in the context of migraine-relevant treatment. As shown in FIGS. 7C and 7D, CSD half-width and amplitude showed more variable serial outcomes than for threshold or propagation rate, independent of the treatment, with no effects of either 100:1 or 30:1 at either 1 hour or 30 minutes pre-administration time.

### Aim 2: Effects of CBD:THC on CSD-Related Head Pain Behavior in WT and FHM1 Mice

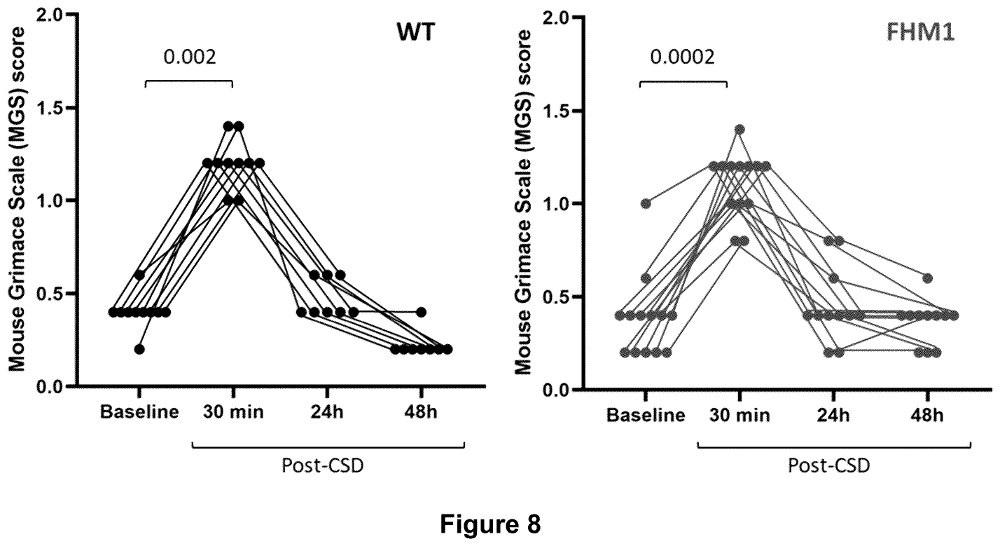

Optogenetically induced CSD causes an acute increase in MGS that has normalized within 24 hours—To test effects of acute pre-administration of CBD:THC on CSD-related head pain behavior, longitudinal DC-EEG recordings were performed for 3-4 weeks with repeated suprathreshold CSD induction and MGS behavioral analysis 30 minutes post-CSD, in mice with minimally-invasive skull electrodes. Before assessing effects of CBD:THC on head pain mimics by MGS scoring, in each mouse we first assessed whether induction of 3 CSDs by optogenetics caused an increase in MGS, assessed from video 30-40 minutes following the first CSD. As shown in FIG. 8, in both WT and FHM1 mice, MGS scores were significantly increased at the 30 minute timepoint after CSD compared to baseline (WT: p=0.002; FHM1: p=0.0002), with post-CSD values typically at a score 1.0 or higher, compared to baseline values typically below 0.6.

Additional tests performed at 24 and 48 hours following CSD showed that both in WT and FHM1 mutant mice MGS scores had returned to baseline already at 24 hours (FIG. 8; p=0.25 for WT, p=0.72 for FHM1). Therefore, drug effects on CSD-related MGS were not assessed for the 24 hour or 48 hour timepoints.

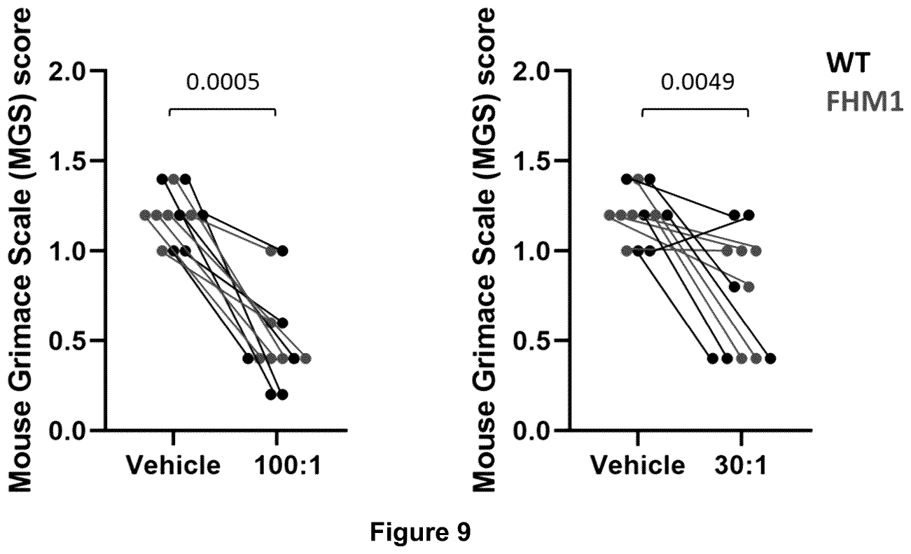

Acute pretreatment by CBD:THC prevents the CSD-induced increase in MGS—As shown in FIG. 9, pretreating mice with an acute dosage of 100:1 or 30:1 CBD:THC, administered i.p. 30 minutes prior to the start of CSD induction, yielded significantly lower MGS scores compared to vehicle treatment.

For 100:1 CBD:THC, effects were significant at the group level of combined WT and FHM1 mice (p=0.0005) as well as for WT and FHM1 groups separate (WT 100:1 vs. vehicle: p=0.031; FHM 100:1 vs vehicle: p=0.031). For the 30:1 dosage, effects were significant at the group level of combined WT and FHM1 mice (p=0.0049) but not for the WT group (N=6; WT 30:1 vs. vehicle: p=0.094) or FHM group separately (N=6; FHM 30:1 vs. vehicle: p=0.063).

### CONCLUSIONS

Aim 1 data showed that CBD:THC, both at a 100:1 and 30:1 ratio, pre-administered either 1 hour or at 30 minutes prior to the experiment, did not affect CSD threshold or other susceptibility features (propagation rate, amplitude, half-width) for optogenetically induced CSD events. Given the knowledge that optogenetic CSD induction relies on ChR2 channel activation, which is a cation channel, it is possible that massive activation of ChR2 channels may not leave enough therapeutic window for assessing effects of CBD:THC, which can act by affecting neuronal ion channel currents.

Aim 2 data showed that CBD:THC has an effect on suppressing CSD-induced head-pain mimics, as evidenced from the significant suppression of opto-CSD induced MGS following acute pretreatment (administration 30 minute prior to CSD induction) with 100:1 CBD:THC for both WT and FHM1 mice. Effects of 30:1 CBD:THC on CSD-MGS were significant as well, but only for the combined WT and FHM1 data-set. These data underscore the potential of CBD:THC as preventive headache treatment, in particular in the context of migraine with aura, i.e. headache following a CSD.

What is claimed is:

1. A method for treating hemiplegic migraine comprising administering a pharmaceutical composition comprising CBD and THC to a subject in need thereof.

2. The method of claim 1, wherein the subject is a male.

3. The method of claim 1, wherein the subject is a human.

4. The method of claim 1, wherein the administering is accomplished via injection.

5. The method of claim 1, wherein the treatment is effective within at least two hours after administration.

6. The method of claim 1, wherein the pharmaceutical composition further comprises a terpenoid.

7. The method of claim 6, wherein the terpenoid comprises myrcene, β-caryophyllene, limonene, α-terpineol, linalool, α-phellandrene, α-pinene, β-pinene, γ-terpinene, nerolidol, phytol, caryophyllene oxide, or α-humule.

8. The method of claim 1, wherein the pharmaceutical composition further comprises an analgesic agent.

9. The method of claim 8, wherein the analgesic agent comprises paracetamol, acetaminophen, a non-steroidal anti-inflammatory drug, a 5-HT (serotonin) receptor agonist, a calcitonin gene related peptide (CGRP) receptor antagonist, an N-methyl D-aspartate (NMDA) inhibitor, a glutamate receptor antagonist, a Substance P inhibitor, a GABA inhibitor, FAAH inhibitor, a Nerve Growth Factor (NGF) inhibitor, a Brain-Derived Neurotrophic Factor (BDNF) inhibitor, an extracellular signal-regulated kinase (ERK) antagonist, or a NO inhibitor.

10. The method of claim 1, wherein the pharmaceutical composition further comprises a pharmaceutically acceptable excipient.

11. The method of claim 10, wherein the pharmaceutically acceptable excipient comprises a carrier, a diluent, a binder, a filler, a buffering agent, a pH modifying agent, a disintegrant, a dispersant, a preservative, a lubricant, a taste-masking agent, a flavoring agent, or a coloring agent.

12. The method of claim 1, wherein the THC and CBD are at least 90 wt. % pure.

13. The method of claim 1, wherein the pharmaceutical composition further comprises at least one minor cannabinoid.

14. The method of claim 13, wherein the at least one minor cannabinoid is selected from the group consisting of CBG, CBGA, CBN, CBNA, THCV, THCA, CBC, CBCA, CBDV, and D8-THC.

15. The method of claim 13, wherein the at least one minor cannabinoid is at least 90 wt. % pure.

16. The method of claim 1, wherein a weight ratio of CBD to THC is from about 50:1 to about 150:1.

17. The method of claim 1, wherein the weight ratio of CBD to THC is from about 75:1 to about 125:1.

18. The method of claim 1, wherein the hemiplegic migraine is familial hemiplegic migraine.

19. The method of claim 1, wherein the hemiplegic migraine is sporadic hemiplegic migraine.

20. The method of claim 1, wherein the pharmaceutical composition is administered upon a first symptom onset of acute attacks of hemiplegic migraine.

21. The method of claim 1, wherein the pharmaceutical composition is administered within 1 hour of the first symptom onset of acute attacks of hemiplegic migraine.

22. The method of claim 1, wherein the subject is free of hemiplegia within 15 minutes, 30 minutes, 1 hour, 4 hours, 24 hours, or 48 hours of the administration.

23. The method of claim 1, wherein the subject experiences reduced arm hemiplegia within 15 minutes, 30 minutes, 1 hour, 4 hours, 24 hours, or 48 hours of the administration.

24. The method of claim 1, wherein the subject experiences reduced leg hemiplegia within 15 minutes, 30 minutes, 1 hour, 4 hours, 24 hours, or 48 hours of the administration.

25. A method for preventing the onset of hemiplegic migraine symptoms comprising administering a pharmaceutical composition comprising at least one minor cannabinoid, CBD, and THC to a subject in need thereof prior to the onset of hemiplegic migraine symptoms.
